(19) United States
(12) Patent Application Publication
(10) Pub. No.: US 2020/0352901 A1
(43) Pub. Date: Nov. 12, 2020

(54) TETRAHYDROCANNABINOL MODULATORS

(71) Applicant: Scientific Holdings, LLC, Monrovia, CA (US)

(72) Inventors: Jeffrey Charles Raber, Monrovia, CA (US); Bradley J. Douglass, Seattle, WA (US)

(73) Assignee: Scientific Holdings, LLC, Monrovia, CA (US)

(21) Appl. No.: 16/957,355

(22) PCT Filed: Sep. 4, 2018
(86) PCT No.: PCT/US18/49360

§ 371 Date: Jun. 23, 2020

Related U.S. Application Data
(60) Provisional application No. 62/553,817, filed on Sep. 2, 2017.

(51) Int. Cl. A61K 31/352; A61K 9/00; A61K 31/05; A61K 38/45

(52) U.S. Cl. CPC A61K 31/352; A61K 38/45; A61K 31/05; A61K 9/0053

(57) ABSTRACT

The disclosure provides cannabinoid compositions that include delta-8-tetrahydrocannabinol (delta-8-THC), cannabidiol (CBD), delta-9-THC, natural products that reduce catabolism of delta-8-THC, delta-8-THC, 11-hydroxy-delta-8-THC, or of 11-hydroxy-delta-9-THC, as well as pharmaceutically synergic or additive combinations of delta-8-THC and delta-9-THC.

### FIELD OF THE DISCLOSURE

The disclosure relates to compositions and methods that provide increased concentrations of active cannabinoids, such as delta-8-tetrahydrocannabinol (delta-8-THC), delta-9-THC, cannabidiol (CBD), and combinations thereof.

### BACKGROUND OF THE DISCLOSURE

The major cannabinoids from Cannabis sativa are cannabidiol (CBD), cannabichromene (CBC), cannabigerol (CBG), delta-9-tetrahydrocannabinol (delta-9-THC), and cannabinol (CBN) (Appendino et al (2008) J. Nat. Prod. 71:1427-1430). Origin of delta-8-tetrahydrocannabinol (delta-8-THC) is described (Owens et al (1981) Clin. Chem. 27:619-624). Regarding another difference between delta-8-THC and delta-9-THC, for treating glaucoma delta-8-THC and delta-9-THC have equivalent efficacy, but delta-8-THC has “little or no central effects” and is “less psychoactive” as compared to delta-9-THC (Marijuana Research Findings:1980, National Institute on Drug Abuse. Research Monograph 31 (R C Peterson, ed.) pages 201-202). Consistently, in a study showing that both delta-8-THC and delta-9-THC are effective as anti-emetics, it was reported that delta-8-THC is “a cannabinoid with lower psychotropic potency than . . . delta-9-THC” (Abrahamov et al (1995) J. Int. Hemp. Assoc. 2:76-79; Abrahamov et al (1995) Life Sciences. 56:2097-2102).

Clinical trials have established that cannabis, or formulations derived from cannabis, can improve neuropathic pain of multiple sclerosis, improve appetite and sleep quality in cancer patients, relieve pain in fibromyalgia patients, and serve as an anti-emetic for chemotherapy induced nausea and vomiting (see, Health Canada (February 2013) Information for Health Care Professionals. Cannabis (Marihuana, Marijuana) and the Cannabinoids (152 pages)). The present disclosure addresses the unmet need for delta-8-THC compositions that have less psychoactive effects than delta-9-THC, and yet are effective for medical effects, such as for treating glaucoma, use as an anti-emetic, increasing restful sleep, use as an anorectant, and so on. Moreover, the present disclosure provides compositions that comprise delta-8-THC that are not detectable by blood or urine tests that detect delta-9-THC or to metabolites of delta-9-THC and are limited by serving size and package limits for delta-9-THC

### SUMMARY OF THE DISCLOSURE

Briefly stated, the present disclosure provides a composition comprising the combination of delta-8-THC and a non-cannabinoid natural product: (i) Wherein the non-cannabinoid natural product is capable of increasing the duration of the psychoactive or the non-psychoactive medicinal effects of delta-8-THC, as determinable by co-administering the delta-8-THC with or without the non-cannabinoid natural product, or (ii) Wherein the non-cannabinoid natural product is capable of increasing the duration of the psychoactive or the non-psychoactive medicinal effects of delta-9-THC, as determinable by co-administering the delta-9-THC with or without the non-cannabinoid natural product, or (iii) Wherein the non-cannabinoid natural product is capable of increasing the concentration of 1-hydroxy-delta-8-THC in the bloodstream of a human subject, as determinable by co-administering delta-8-THC with or without the non-cannabinoid natural product, or (iv) Wherein the non-cannabinoid natural product is capable of increasing the concentration of 1l-hydroxy-delta-9-THC in the bloodstream as determinable by co-administering delta-9-THC with or without the non-cannabinoid natural product to the human subject.

The present disclosure also embraces the above composition that further comprises delta-9-THC, or the above composition that does not comprise delta-9-THC. Moreover, what is provided is the above composition wherein the cannabinoid and non-cannabinoid natural product are mixed together as a pharmaceutically acceptable composition for oral administration, where optionally the pharmaceutically acceptable composition for oral administration is a powder, tablet, pill, capsule, slurry, suspension, or liquid composition.

Also contemplated is the above composition, wherein the delta-8-THC and non-cannabinoid natural product that are not mixed together, wherein the delta-8-THC is a component of a pharmaceutically acceptable composition for oral administration, and wherein the non-cannabinoid is a component of a pharmaceutically acceptable composition for oral administration.

Additionally, the disclosure provides the above composition, that further comprises an inhibitor of at least one UDP-glucuronosyl transferase (UGT), wherein the UGT in absence of inhibitor is capable of catalyzing glucuronidation of one or both 11-hydroxy-delta-8-THC and 11-hydroxy-delta-9-THC, where optionally the inhibitor is a substrate of UGT that is capable of acting as a competitive inhibitor of the at least one UGT. Also encompassed is the above composition, that further comprises an inhibitor of at least one UDP-glucuronosyl transferase (UGT), wherein the UGT in absence of inhibitor is capable of catalyzing glucuronidation of one or both 11-hydroxy-delta-8-THC and 11-hydroxy-delta-9-THC, wherein the inhibitor comprises one or more of curcumin, carvacrol, and nor-oleanane triterpenoid saponin.

Further encompassed is the above composition, that further comprises an inhibitor of a cytochrome P450 enzyme (CYP enzyme), wherein the CYP enzyme catalyzes the metabolism of a psychoactive cannabinoid to a non-psychoactive metabolite, or wherein the CYP enzyme catalyzes the metabolism of a non-psychoactive medically active cannabinoid to a non-psychoactive non-medically active metabolite.

Additionally provided is the above composition that comprises an inhibitor of an alcohol dehydrogenase that catalyzes conversion of 11-hydroxy-delta-9-THC to the corresponding carboxyaldehyde. Also, what is provided is the above composition, that comprises an inhibitor of an aldehyde dehydrogenase or an aldehyde oxidase that catalyzes conversion of the carboxyaldehyde of 11-hydroxy-delta-9-THC to 11-nor-9-carboxy-delta-9-THC. Further contemplated is the above composition that comprises an inhibitor of an alcohol dehydrogenase that catalyzes conversion of 11-hydroxy-delta-8-THC to the corresponding carboxyaldehyde.

In yet another aspect, what is provided is the above composition, that comprises an inhibitor of an aldehyde dehydrogenase or an aldehyde oxidase that catalyzes conversion of the carboxyaldehyde of 1-hydroxy-delta-8-THC to 11-nor-9-carboxy-delta-8-THC. Moreover, what is provided is the above composition, that comprises an inhibitor that inhibits CYP3A4-mediated conversion of delta-8-THC to 7-hydroxy-delta-8-THC.

In yet another aspect, the disclosure contemplates the above composition that comprises an inhibitor that inhibits CYP3A4-mediated conversion of delta-8-THC to 7-hydroxy-delta-8-THC, wherein the inhibitor comprises one or more of grapefruit juice, bergamottin, peppermint oil, a sesquiterpene, and a curcuminoid.

In psychoactive embodiments, the disclosure provides the above composition, wherein the psychoactive effects comprise one or more of: (i) Decreased rapid eye movement (REM) sleep; (ii) Increased deep sleep; or (iii) Reduced seizure rate or seizure intensity. In non-psychoactive embodiments, what is provided is the above composition, wherein the non-psychoactive medical effects comprise one or more of: (i) Anti-emetic effect; (ii) Neuroprotectant effect; or (iii) Anorectant effect.

In cannabinoid receptor embodiments, the present disclosure provides a pharmaceutically acceptable composition capable of oral administration to a human subject, the composition comprising delta-8-THC and delta-9-THC, wherein (i) The administered composition results in stimulation of CB1, or (ii) The administered composition results in stimulation of CB2, or (iii) The administered composition results in stimulation of CB1 to a greater extent than administration of delta-8-THC alone, or (iv) The administered composition results in stimulation of CB1 to a greater extent than administration of delta-9-THC alone, or (v) The administered composition results in stimulation of CB2 to a greater extent than administration of delta-8-alone, or (vi) The administered composition results in stimulation of CB2 to a greater extent than administration of delta-9-THC alone, (vii) The delta-8-THC in the administered composition enhances the pharmacological activity of the delta-9-THC in the administered composition, or (viii) The delta-9-THC in the administered composition enhances the pharmacological activity of the delta-8-THC in the administered composition.

Very high amount ranges are provided. What is provided is the above pharmacologically acceptable composition that comprises a tablet containing over 30 mg of delta-8-THC and 10-30 mg of delta-9-THC, or a first tablet containing over 30 mg of delta-8-THC and a second tablet containing 10-30 mg of delta-9-THC. Provided is the above pharmacologically acceptable composition that comprises a tablet containing over 30 mg of delta-8-THC and 2-10 mg of delta-9-THC, or a first tablet containing over 30 mg of delta-8-THC and a second tablet containing 2-10 mg of delta-9-THC. Provided is the above pharmacologically acceptable composition that comprises a tablet containing over 30 mg of delta-8-THC and 0.5-2.0 mg of delta-9-THC, or a first tablet containing over 30 mg of delta-8-THC and a second tablet containing 0.5-2.0 mg of delta-9-THC. Also encompassed, is the above pharmacologically acceptable composition that comprises a tablet containing over 30 mg of delta-8-THC and 0.01-0.5 mg of delta-9-THC, or a first tablet containing over 30 mg of delta-8-THC and a second tablet containing 0.01-0.5 mg of delta-9-THC. What is also provided are the above compositions, where each quantity is preceded by the word, “about.” In addition to tablet embodiments, what is also provided are pills, capsules, powders (e.g., first powder and a second powder), gels, lotions, slurries, liquids, aerosols, and so on.

High amount ranges are provided. What is provided is the above pharmacologically acceptable composition that comprises a tablet containing 10-30 mg of delta-8-THC and 10-30 mg of delta-9-THC, or a first tablet containing 10-30 mg of delta-8-THC and a second tablet containing 10-30 mg of delta-9-THC. Provided is the above pharmacologically acceptable composition that comprises a tablet containing 10-30 mg of delta-8-THC and 2-10 mg of delta-9-THC, or a first tablet containing 10-30 mg of delta-8-THC and a second tablet containing 2-10 mg of delta-9-THC. Provided is the above pharmacologically acceptable composition that comprises a tablet containing 10-30 mg of delta-8-THC and 0.5-2.0 mg of delta-9-THC, or a first tablet containing 10-30 mg of delta-8-THC and a second tablet containing 0.5-2.0 mg of delta-9-THC. Also encompassed, is the above pharmacologically acceptable composition that comprises a tablet containing 10-30 mg of delta-8-THC and 0.01-0.5 mg of delta-9-THC, or a first tablet containing 10-30 mg of delta-8-THC and a second tablet containing 0.01-0.5 mg of delta-9-THC. What is also provided are the above compositions, where each quantity is preceded by the word, “about.”

Medium amount ranges are provided. What is provided is the above pharmacologically acceptable composition that comprises a tablet containing 10 mg of delta-8-THC and 10 mg of delta-9-THC, or a first tablet containing 10 mg of delta-8-THC and a second tablet containing 10 mg of delta-9-THC. Provided is the above pharmacologically acceptable composition that comprises a tablet containing 1.0 mg of delta-8-THC and 1.0 mg of delta-9-THC, or a first tablet containing 1.0 mg of delta-8-THC and a second tablet containing 1.0 mg of delta-9-THC. Provided is the above pharmacologically acceptable composition that comprises a tablet containing 1.0 mg of delta-8-THC and 0.5 mg of delta-9-THC, or a first tablet containing 1.0 mg of delta-8-THC and a second tablet containing 0.5 mg of delta-9-THC. Also encompassed, is the above pharmacologically acceptable composition that comprises a tablet containing 1.0 mg of delta-8-THC and 0.25 mg of delta-9-THC, or a first tablet containing 1.0 mg of delta-8-THC and a second tablet containing 0.25 mg of delta-9-THC. Also embraced, is the above pharmacologically acceptable composition that comprises a tablet containing 1.0 mg of delta-8-THC and 0.125 mg of delta-9-THC, or a first tablet containing 1.0 mg of delta-8-THC and a second tablet containing 0.125 mg of delta-9-THC. What is also provided are the above compositions, where each quantity is preceded by the word, “about.”

Low amount ranges are provided. In embodiments with still lower quantities of delta-9-THC, what is provided is the above pharmacologically acceptable composition that comprises a tablet containing 4.0 mg of delta-8-THC and 0.125 mg of delta-9-THC, or a first tablet containing 4.0 mg of delta-8-THC and a second tablet containing 0.125 mg of delta-9-THC, or a tablet containing 2.0 mg of delta-8-THC and 0.125 mg of delta-9-THC, or a first tablet containing 2.0 mg of delta-8-THC and a second tablet containing 0.125 mg of delta-9-THC, or a tablet containing 1.0 mg of delta-8-THC and 0.125 mg of delta-9-THC, or a first tablet containing 1.0 mg of delta-8-THC and a second tablet containing 0.125 mg of delta-9-THC. What is also provided are the above compositions, where each quantity is preceded by the word, “about.”

Very low amount ranges are provided. Provided is the above pharmacologically acceptable composition that comprises a tablet containing 2.0 mg of delta-8-THC and 2.0 mg of delta-9-THC, or a first tablet containing 2.0 mg of delta-8-THC and a second tablet containing 2.0 mg of delta-9-THC, a tablet containing 2.0 mg of delta-8-THC and 1.0 mg of delta-9-THC, or a first tablet containing 2.0 mg of delta-8-THC and a second tablet containing 1.0 mg of delta-9-THC. Provided is the above pharmacologically acceptable composition that comprises a tablet containing 2.0 mg of delta-8-THC and 0.5 mg of delta-9-THC, or a first tablet containing 2.0 mg of delta-8-THC and a second tablet containing 0.5 mg of delta-9-THC. Also encompassed, is the above pharmacologically acceptable composition that comprises a tablet containing 2.0 mg of delta-8-THC and 0.25 mg of delta-9-THC, or a first tablet containing 2.0 mg of delta-8-THC and a second tablet containing 0.25 mg of delta-9-THC. What is also provided are the above compositions, where each quantity is preceded by the word, “about.” In addition to tablet embodiments, what is also provided are pills, capsules, powders (e.g., first powder and a second powder), gels, lotions, slurries, liquids, aerosols, and so on.

Range embodiments are also provided, where the range can consist of any two adjacent values, or any three consecutive adjacent values, or any four consecutive adjacent values, and so on. For example, for the above disclosure of: “Provided is the above pharmacologically acceptable composition that comprises a tablet containing 2.0 mg of delta-8-THC and 2.0 mg of delta-9-THC, . . . a tablet containing 2.0 mg of delta-8-THC and 1.0 mg of delta-9-THC,” the range embodiment would be, “a tablet containing 2.0 mg of delta-8-THC and 1.0 to 2.0 mg of delta-9-THC.”

Also provided is the above pharmaceutically acceptable composition that is capable of one or more of oral administration, intranasal administration, mucosal administration, or administration by inhaling, to a human subject.

Moreover, in yet another aspect what is provided is the above pharmaceutically acceptable composition, wherein the greater extent of stimulation is determinable by comparing stimulation of the CB1 or of the CB2 by: (a) Administering the composition comprising delta-8-THC and delta-9-THC, with (b) Administering delta-8-THC in an amount equivalent to that present in the composition. Furthermore, what is provided is the above pharmaceutically acceptable composition, wherein the greater extent of stimulation is determinable by comparing stimulation of the CB1 or of the CB2 by: (a) Administering the composition comprising delta-8-THC and delta-9-THC, with (b) Administering delta-9-THC in an amount equivalent to that present in the composition.

In an embodiment that extrapolates animal cannabinoid receptor data to human cannabinoid receptors, the disclosure provides the above pharmaceutically acceptable composition, wherein the stimulation of CB1 and the stimulation of CB2 in human subjects is determinable by administering to an animal subject a composition comprising delta-8-THC and delta-9-THC, by administering delta-8-alone, and by administering delta-9-alone, and by extrapolating the stimulation results to humans.

Methods for administration of the above compositions are also provided, for example, comprising the step of providing a compound, or alternatively, the step of providing a first compound and a second compound, further comprising the step of oral administration (of a compound, or of both a first compound and a second compound), the step of administering by nasal inhalation (of a compound, or of both a first compound and a second compound), the step of oral administration combined with nasal inhalation (both for one compound), or alternatively, the step of oral administration (for a first compound) and the step of nasal administration (for a second compound).

Also, methods for manufacturing the above compositions are contemplated. Cannabidiol (CBD) embodiments are also provided.

The present disclosure provides a composition comprising the combination of delta-8-THC, cannabidiol (CBD), and a non-cannabinoid natural product: (i) Wherein the non-cannabinoid natural product is capable of increasing the duration of the psychoactive or the non-psychoactive medicinal effects of delta-8-THC, as determinable by co-administering the delta-8-THC with or without the non-cannabinoid natural product, or (ii) Wherein the non-cannabinoid natural product is capable of increasing the duration of the psychoactive or the non-psychoactive medicinal effects of CBD, as determinable by co-administering the CBD with or without the non-cannabinoid natural product, or (iii) Wherein the non-cannabinoid natural product is capable of increasing the concentration of 11-hydroxy-delta-8-THC in the bloodstream of a human subject, as determinable by co-administering delta-8-THC with or without the non-cannabinoid natural product, or (iv) Wherein the non-cannabinoid natural product is capable of increasing the concentration of 11-hydroxy-CBD in the bloodstream as determinable by co-administering CBD with or without the non-cannabinoid natural product to the human subject.

Also, provided is the above composition that further comprises delta-9-THC. Moreover, what is provided is the above composition that does not comprise delta-9-THC. In addition, what is provided is the above composition, wherein the delta-8-THC, cannabidiol (CBD), and non-cannabinoid natural product, are mixed together as a pharmaceutically acceptable composition for oral administration, where optionally the pharmaceutically acceptable composition for oral administration is a powder, tablet, pill, capsule, slurry, suspension, or liquid composition.

Moreover, what is provided is the above composition, wherein the delta-8-THC, CBD, and non-cannabinoid natural product that are not all mixed together, wherein the delta-8-THC is a component of a first pharmaceutically acceptable composition for oral administration, wherein the CBD is a component of a second pharmaceutically acceptable composition for oral administration, and wherein the non-cannabinoid is a component of a third pharmaceutically acceptable composition for oral administration. Alternatively, the delta-8-THC and CBD can be provided together in a fourth pharmaceutically acceptable composition. Also, the delta-8-THC and non-cannabinoid natural product can be provided together in a fifth pharmaceutically acceptable composition. Moreover, the CBD and the non-cannabinoid natural product can be provided together in a sixth pharmaceutically acceptable composition.

Also contemplated, as the above composition that further comprises an inhibitor of at least one UDP-glucuronosyl transferase (UGT), wherein the UGT in absence of inhibitor is capable of catalyzing glucuronidation of one or both 11-hydroxy-delta-8-THC and CBD, where optionally the inhibitor is a substrate of UGT that is capable of acting as a competitive inhibitor of the at least one UGT. In another aspect, what is provided is the above composition, that further comprises an inhibitor of at least one UDP-glucuronosyl transferase (UGT), wherein the UGT in absence of inhibitor is capable of catalyzing glucuronidation of one or both 11-hydroxy-delta-8-THC and CBD, wherein the inhibitor comprises one or more of curcumin, carvacrol, and nor-oleanane triterpenoid saponin.

Further contemplated, is the above composition, that further comprises an inhibitor of a cytochrome P450 enzyme (CYP enzyme), wherein the CYP enzyme catalyzes the metabolism of a psychoactive cannabinoid to a non-psychoactive metabolite, or wherein the CYP enzyme catalyzes the metabolism of a non-psychoactive medically active cannabinoid to a non-psychoactive non-medically active metabolite. Also available, is the above composition, that comprises an inhibitor of an alcohol dehydrogenase that catalyzes conversion of 11-hydroxy-CBD to the corresponding carboxyaldehyde. Further embraced, is the above composition, that comprises an inhibitor of an aldehyde dehydrogenase or an aldehyde oxidase that catalyzes conversion of the carboxyaldehyde of 11-hydroxy-CBD to 11-nor-9-carboxy-CBD. Additionally, what is provided is the above composition, that comprises an inhibitor of an alcohol dehydrogenase that catalyzes conversion of 11-hydroxy-delta-8-THC to the corresponding carboxyaldehyde. In yet another aspect, what is provided is the above composition, that comprises an inhibitor of an aldehyde dehydrogenase or an aldehyde oxidase that catalyzes conversion of the carboxyaldehyde of 11-hydroxy-delta-8-THC to 11-nor-9-carboxy-delta-8-THC.

Also embraced, is the above composition that comprises an inhibitor that inhibits CYP3A4-mediated conversion of delta-8-THC to 7-hydroxy-delta-8-THC. Moreover, what is provided is the above composition, that comprises an inhibitor that inhibits CYP3A4-mediated conversion of delta-8-THC to 7-hydroxy-delta-8-THC, wherein the inhibitor comprises one or more of grapefruit juice, bergamottin, peppermint oil, a sesquiterpene, and a curcuminoid.

In psychoactive and medical effect embodiments, what is provided is the above composition, wherein the psychoactive effects comprise one or more of: (i) Decreased rapid eye movement (REM) sleep; (ii) Increased deep sleep; or (iii) Reduced seizure rate or seizure intensity. Also provided, is the above composition, wherein the non-psychoactive medical effects comprise one or more of (i) Anti-emetic effect; (ii) Neuroprotectant effect; or (iii) Anorectant effect.

In CB1 and CB2 embodiments, what is provided is a pharmaceutically acceptable composition capable of oral administration to a human subject, the composition comprising delta-8-THC and cannabinol (CBD), wherein (i) The administered composition results in stimulation of CB1, or (ii) The administered composition results in stimulation of CB2, or (iii) The administered composition results in stimulation of CB1 to a greater extent than administration of delta-8-THC alone, or (iv) The administered composition results in stimulation of CB1 to a greater extent than administration of CBD alone, or (v) The administered composition results in stimulation of CB2 to a greater extent than administration of delta-8-THC alone, or (vi) The administered composition results in stimulation of CB2 to a greater extent than administration of CBD alone, (vii) The delta-8-THC in the administered composition enhances the pharmacological activity of the delta-9-THC in the administered composition, or (viii) The CBD in the administered composition enhances the pharmacological activity of the delta-8-THC in the administered composition.

In combination embodiments that provide both delta-8-THC and CBD, what is provided is the above pharmacologically acceptable composition of that comprises a tablet containing delta-8-THC and CBD in the amounts: (i) 10 mg of delta-8-THC and 10 mg of CBD, or (ii) 5 mg delta-8-THC and 5 mg CBD, or (iii) 2 mg delta-8-THC and 2 mg CBD, or (iv) 1 mg delta-8-THC and 1 mg CBD, or (v) 5 mg delta-8-THC and 2 mg CBD, or (vi) 5 mg delta-8-THC and 1 mg CBD, or (vii) 5 mg delta-8-THC and 0.5 mg CBD, or (viii) 2 mg delta-8-THC and 1 mg CBD, or (ix) 2 mg delta-8-THC and 0.5 mg CBD, or (x) 2 mg delta-8-THC and 0.25 mg CBD, or (xi) 1 mg delta-8-THC and 1 mg CBD, or (xii) 1 mg delta-8-THC and 0.5 mg CBD, or (xiii) 1 mg delta-8-THC and 0.25 mg CBD, or containing delta-8-THC and CBD in approximately said amounts.

Moreover, what is embraced is the above pharmaceutically acceptable composition of that is capable of one or more of oral administration, intranasal administration, mucosal administration, or administration by inhaling, to a human subject. Also provided is the above pharmaceutically acceptable composition, wherein the greater extent of stimulation is determinable by comparing stimulation of the CB1 or of the CB2 by: (a) Administering the composition comprising delta-8-THC and CBD, with (b) Administering delta-8-THC in an amount equivalent to that present in the composition. Moreover, what is contemplated is the above pharmaceutically acceptable composition, wherein the greater extent of stimulation is determinable by comparing stimulation of the CB1 or of the CB2 by: (a) Administering the composition comprising delta-8-THC and CBD, with (b) Administering CBD in an amount equivalent to that present in the composition. In yet another aspect, what is provided is the above pharmaceutically acceptable composition of, wherein the stimulation of CB1 and the stimulation of CB2 in human subjects is determinable by administering to an animal subject a composition comprising delta-8-THC and CBD, by administering delta-8-alone, and by administering CBD alone, and by extrapolating the stimulation results to humans.

In screening methods embodiment, the present disclosure provides a method for screening non-cannabinoid natural products to identify a pharmaceutically acceptable non-cannabinoid natural product that is capable of increasing the concentration of a biologically active cannabinoid in a biological fluid of a test mammal, or reducing the concentration of a biologically inactive cannabinoid in a biological fluid of a test mammal, the method comprising: (i) Administering delta-8-THC plus cannabidiol (CBD) to the test mammal, (ii) Co-administering the non-cannabinoid natural product to the test mammal, where a first period of time is required to initiate and complete administering of the delta-8-THC plus CBD, and where a second period of time is required to initiate and complete administering the non-cannabinoid natural product, (iii) Where the first period of time is identical to the second period of time, where the first period of time overlaps but is not identical to the second period of time, or where the first period of time does not overlap the second period of time, (iv) After the completion of both the first period of time and the second period of time, and within five days of completion of both the first period of time and the second period of time, taking at least one sample of the biological fluid from the test mammal and transferring the sample to a container, (v) Subjecting the sample to a detection method that is capable of detecting one or more of the biologically active compounds delta-8-THC, 11-hydroxy-delta-8-THC, CBD, 11-hydroxy-CBD, 7-hydroxy-delta-8-THC, 7-hydroxy-CBD, or that is capable of detecting one or more biologically inactive compounds, 11-nor-9-carboxy-delta-8-THC, 11-nor-9-carboxy-CBD, 7-hydroxy-delta-8-THC, or 7-hydroxy-CBD, (vi) Detecting said one or more biologically active compounds and biologically inactive compounds and calculating the concentration of said one or more compounds in the biological fluid.

As an alternative to the above-disclosed, “After the completion of both the first period of time and the second period of time, and within five days of completion of both the first period of time and the second period of time,” the method provides embodiments of, within one day, within two days, within three days, within four days, within six days, within seven days, within eight days, within nine days, within ten days, within 1 week, within 2 weeks, within 3 weeks, within 4 weeks, and so on.

Moreover, what is provided is the above method, further comprising administering delta-8-THC to a control mammal, refraining from co-administering the non-cannabinoid natural product, taking at least one sample of the biological fluid from the mammal within five days of administering the delta-8-THC and transferring the sample to a container, and subjecting the sample to a detection method that is capable of detecting one or more of compounds delta-8-THC, 11-hydroxy-delta-8-THC, CBD, 11-hydroxy-CBD, 7-hydroxy-delta-8-THC, 7-hydroxy-CBD, 11-nor-9-carboxy-delta-8-THC, 11-nor-9-carboxy-CBD, and detecting said one or more compounds and calculating the concentration of said one or more compounds in the biological fluid, comparing the concentration from the control mammal with the concentration from the test mammal, and determining the extent that the non-cannabinoid influences the concentration of the one or more compounds.

In further versions of the screening embodiment, what is provided is the above method, wherein the test mammal is human subject and wherein the control mammal is a human subject. Also provided is the above method, wherein the test mammal is human subject, wherein the control mammal is a human subject, and wherein the test mammal is the same human subject as the control mammal.

Biological fluid embodiments are contemplated. Additionally, what is provided is the above method, wherein the biological fluid is blood plasma, whole blood, blood serum (serum), urine, saliva, mucus, sweat, semen, cerebrospinal fluid, and so on. Moreover, what is encompassed is tissue samples, such as hair, skin, liver biopsy, and such. Analytical methods are provided (see, e.g., White R M (2017) Drugs in hair. Part I Metabolisms of major drug classes. Forensic Sci. Rev. 29:23-55. Beasley E et al (2016) Detection and mapping of cannabinoids in single hair samples through rapid derivatization and matrix-assisted laser desorption ionization mass spectrometry. Anal. Chem. 88:10328-10334. Gambelunghe C et al (2016) Cannabis use surveillance by sweat analysis. Ther. Drug Monit. 38:634-639.).

Moreover, what is provided is the above method, wherein the pharmaceutically acceptable natural product is orally administered and the delta-8-THC is orally administered, or wherein the pharmaceutically acceptable non-cannabinoid natural product is orally administered and the CBD is orally administered, or wherein the pharmaceutically acceptable non-cannabinoid natural product is orally administered and the delta-8-THC and the CBD is orally administered.

In yet another aspect, what is provided is the above method, wherein the pharmaceutically acceptable non-cannabinoid natural product comprises one or more of a terpene, carvecrol, curcumin, CYP enzyme inhibitor, and a UGT enzyme inhibitor.

In administering methods embodiments, what is provided is a method for administering one of the above the compositions to a human subject, comprising the steps of: (i) Providing said composition to the human subject, (ii) Administering said composition to the human subject, or self-administering said composition by the human subject, (iii) Allowing a cannabinoid of the composition to increase in concentration in the bloodstream of said human subject, and (iv) Wherein said administering results in a psychological or medical influence on said human subject, assessing the influence by one or both of a questionnaire or a biochemical test. Provided is the above administering method embodiment, that comprises oral administration, or that comprises nasal administration, or that comprises mucosal administration (e.g., intranasal formulation or a suppository), or that comprises administration by inhalation, or that comprises topical administration, or that comprises any combination thereof. Topical administration can use a skin patch (see, U.S. Pat. Nos. 6,444,454, 7,54,190, 8,151,987, and 8,840,921, each of which is incorporated herein by reference, in its entirety) or it can be via skin lotion or skin cream.

Compositions that increase bloodstream concentrations of delta-8-THC or active metabolites thereof, or that increase bloodstream concentrations of cannabinol or active metabolites thereof, are provided. Active cannabinoids, and metabolites thereof, have been established by the literature, and these include those with psychoactive effects, non-psychoactive medical effects, and those with both psychoactive and medical effects.

What is provided is a composition comprising the combination of delta-8-THC and a non-cannabinoid natural product, wherein the non-cannabinoid natural product is capable of increasing the concentration of 11-hydroxy-delta-8-THC in the bloodstream of a human subject, as determinable by in vitro tests capable of detecting the ability of the non-cannabinoid natural product to inhibit CYP enzyme-mediated catabolism of the delta-8-THC (or an active metabolite thereof) to an inactive product, or capable of detecting the ability of the non-cannabinoid natural product to inhibit UDP-glucuronosyl transferase (UGT)-mediated catabolism of the delta-8-THC (or an active metabolite thereof) to an inactive product. Also, what is provided is a composition comprising the combination of cannabidiol (CBD) and a non-cannabinoid natural product, wherein the non-cannabinoid natural product is capable of increasing the concentration of 11-hydroxy-CBD in the bloodstream.

as determinable by in vitro tests capable of detecting the ability of the non-cannabinoid natural product to inhibit CYP enzyme mediated catabolism of the CBD (or an active metabolite thereof) to an inactive product, or capable of detecting the ability of the non-cannabinoid natural product to inhibit UDP-glucuronosyl transferase (UGT) mediated catabolism of the CBD (or an active metabolite thereof) to an inactive product.

Embodiments encompassing a family of THC isomers is encompassed. What is provided is a composition comprising one or more of delta-8-THC, cannabidiol (CBD), delta-7-THC, delta-10-THC, or a cannabinoid where a double bond is present at a ring carbon other than at the 8-position or 9-position, wherein the composition provides an amount of delta-9-THC that is equal or less than a defined maximal amount of delta-9-THC, and wherein: (i) The composition comprises delta-9-THC; or (ii) The composition comprises a non-cannabinoid natural product that is capable of modulating the activity of a cytochrome P450 (CYP) enzyme in a human subject resulting in a CYP enzyme with modulated activity, and wherein the modulated activity results in increased in vivo concentrations in the human subject of an active metabolite of the administered delta-8-THC, cannabidiol (CBD), delta-7-THC, or delta-10-THC, or other similar THC isomer; or (iii) The composition comprises a non-cannabinoid natural product that is capable of inhibiting the activity of UDP-glucuronosyl transferase (UGT), and wherein the inhibited UGT results in increased in vivo concentrations in the human subject of an active metabolite of the administered delta-8-THC, cannabidiol (CBD), delta-7-THC, or delta-10-THC, or other similar THC isomer; or (iv) The cannabinoid where a double bond is present at a ring carbon other than at the 8-position or 9-position is not delta-7-THC or delta-10-THC.

Embodiments encompassing alternative double bond positions are provided. What is provided is a cannabinoid where a double bond is present at a ring carbon other than at the 8-position or 9-position is not delta-7-THC or delta-O-THC, but still yields an active metabolite, and where the double bond at the ring carbon other than at the 8-position or 9-position is between carbons 9 and 11 (double bond on 11-methyl), carbons 7 and 6a, carbons 6a and 6, carbons 6 and 12 (double bond on 12-methyl), 6 and 13 (double bond on 13-methyl), carbons 10 and 10a, carbons 6a and 10a, and carbons 10a and 10b. Also encompassed, are cannabinoids with more than one double bond, and where the bonds are at the indicated position. What can be excluded are compositions and methods, with where the cannabinoid has a double bond at one or more of the above position.

In some embodiments, what is provided is the cannabinoid where the double bond is a cis double bond, while in other aspects the double bond is a trans double bond. Also, embraced is a cannabinoid with a plurality of double bonds, where all of the double bonds are cis, where all of the double bonds are trans, or where one is cis and the other is trans, or where some are cis and the others are trans.

Further double bond position embodiments, are cannabinoids with a double bond between carbons 9 and 10, between carbons 8 and 9, between carbons 9 and 11 (double bond on 11-methyl group), between carbons 7 and 6a, between carbons 6a and 6, between carbons 6 and 12 (double bond on 12-methyl group), between carbons 6 and 13 (double bond on 13-methyl group), between carbons 10 and 10a, between carbons 6a and 10a, or between carbons 10a and 10b.

In some aspects, what is provided is the cannabinoid where the double bond is a cis double bond, while in other aspects the double bond is a trans double bond. Also, contemplated is a cannabinoid with a plurality of double bonds, where all of the double bonds are cis, where all of the double bonds are trans, or where one is cis and the other is trans, or where some are cis and the others are trans. In some aspects, these cannabinoids do not have any double bond at the 8-position, or do not have any double bound at the 9-position, or do not have any double bond at the 8-position or 9-position.

Monohydroxy derivatives may include a cannabinoid with a hydroxyl group on carbon number, 1, 2, 3, 4, 5, 6, 6a, 7, 8, 9, 10, 11 (methyl group), 12 (methyl group), 13 (methyl group), 1′, 2′, 3′, 4′, and 5′. Encompassed are cannabinoids with a plurality of hydroxyl groups at a plurality of carbon positions. Also, encompassed are cannabinoids with two hydroxyl groups on a given carbon group. Also, what can be excluded is any composition or method that has one or more of the above monohydroxyl derivatives. Numbering according to, Pertwee R G et al (2010) International Union of Basic and Clinical Pharmacology. LXXIX. Cannabinoid receptors and their ligands: beyond CB1 and CB1. Pharmacol. Rev. 62:588-631.

Also provided is the above composition, wherein said active metabolite is one or more of psychoactive, medically active, and pharmacologically active.

Compositions, and related methods, that are limited by laws or by sports regulations are encompassed. What is provided is any of the compositions disclosed above, wherein the defined maximal concentration of delta-9-THC is defined by one or both of: (i) law by the State of Washington, the State of Oregon, the State of California, or the State of Colorado, or any other states or jurisdictions with similarly defined laws, or (ii) Drug testing policy by the National Football League or other professional or non-professional sport governing bodies.

Compositions, and related methods, that are limited by cannabinoid concentration, such as mg/L, micromolar, and nanograms/mg tissue, are provided. What is provided is any one or more of the above compositions, wherein the defined maximal concentration of delta-9-THC, or its signaling metabolites, is an amount detectable in whole blood, in blood plasma, in urine, or in other bodily fluids, of the human subject. Also provided is the above composition, wherein the defined maximal concentration is equal or less than 10 nanograms (ng) per mL, equal or less than 5 ng per mL, equal or less than 2 ng per mL, or equal or less than 1 ng per mL. Also provided is the above composition, wherein the maximal amount of delta-9-THC is 1 mg delta-9-THC, 2 mg delta-9-THC, 5 mg delta-9-THC, or 10 mg delta-9-THC. In another aspect, what is provided is the above composition, comprising one or more of delta-8-THC, cannabidiol (CBD), delta-7-THC, or delta-10-THC, wherein the delta-7-THC possesses psychoactive or medicinal activity and wherein said activity is exerted by 11-hydroxy-delta-7-THC, or where in the delta-10-THC possesses psychoactive or medicinal activity, and wherein said activity is exerted by 11-hydroxy-delta-10-THC, or wherein other similar isomers possess psychoactive or medicinal activity, and wherein said activity is exerted by the mono-hydroxy metabolites of such isomers. In yet another aspect, what is provided is the above composition, that is a single serving composition, as well as the above composition that is not a single serving composition.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference.

Concentrations and Amounts of THC Compounds

Washington State Liquor and Cannabis Board (WSLCB) has set forth limits to the concentration of delta-9-THC in the bloodstream, for use in determining driving under the influence (DUI): “What is the DUI provision? The initiative sets a per se DUI limit of “delta-9” THC levels at greater than or equal to 5 nanograms per milliliter of blood (5 ng/mL). State and local law enforcement agencies are tasked with enforcing the DUI limit.” (accessed Aug. 3, 2017). Regarding testing, a publication from NTSA states, “Of special interest was marijuana use by drivers. We tested for the psychoactive substance delta-9-tetrahydrocannabinol, commonly known as THC; the active metabolite 11-hydroxy-delta-9-tetrahydrocannabinol (also noted as 11-OH-THC and known as “hydroxyTHC”); and the inactive metabolite 11-nor-9-carboxy-delta-9-tetrahydrocannabinol (also known as “carboxy-THC” and noted as “THC-COOH”).” (U.S. Dept. Transportation. National Traffic Safety Administration (NTSA) (July 2016) Marijuana, Other Drugs, and Alcohol Use by Drivers (73 pages). Also, the NTSA publication refers to legal limits of THC in blood, “In December 2012, Washington began implementing the provisions of legalization, which included . . . amendment of the State's driving under the influence statutes to include a per se limit for THC (5 ng/mL).”

A key component of most DUI provisions and other testing provisions use to establish the consumption of cannabis or cannabis products, define “THC” narrowly to only include delta-9 THC and therefore a positive test is based soley upon levels of delta-9 THC metabolites not the metabolites of any other isomers.

The present disclosure provides compositions that provide no detectable increase in blood levels of delta-9-THC (or levels of metabolites of delta-9-THC) as compared to baseline level in absence of administration of the composition. What is compared is delta-9-THC concentrations for a given human subject, where the subject is known not to have consumed (or inhaled) any source of THC (baseline), and where the subject has consumed a composition of the present disclosure. For the baseline measurement, the human subject may be one who has never consumed (or inhaled) any source of THC, or one who has not consumed any source of THC within the previous five weeks.

The present disclosure provides compositions and methods, resulting in Cmax of a given cannabinoid, where the Cmax in whole blood is less than a given concentration such as 5 ng/mL, where the Cmax in blood plasma is less than a given concentration such as 5 mg/mL, or where the Cmax in blood serum is less than a given concentration such as 5 mg/mL.

Also provided, are compositions that provide an increase in detectable blood levels of delta-9-THC to a maximal concentration (Cmax), and where the Cmax is less than 5 ng/mL, less than 4.8 ng/mL, less than 4.6 ng/mL, less than 4.4 ng/mL, less than 4.2 ng/mL, less than 4.0 ng/mL, less than 3.8 ng/mL, less than 3.6 ng/mL, less than 3.4 ng/mL, less than 3.2 ng/mL, less than 3.0 ng/mL, less than 2.8 ng/mL, less than 2.6 ng/mL, less than 2.4 ng/mL, less than 2.2 ng/mL, less than 2.0 ng/mL, less than 1.8 ng/mL, less than 1.6 ng/mL, less than 1.4 ng/mL, less than 1.2 ng/mL, less than 1.0 ng/mL, and the like.

As an alternative to the bloodstream concentration parameter Cmax, the parameter of area under the curve (AUC) can be used. AUC refers to the integrated area of blood concentration, as compared to a baseline concentration level, over a given period of time. The given period of time can be AUC 0-24 hours, or AUC 0 hours-infinity, and so on.

In alternative embodiments, the concentration limits are those from human urine, human saliva, or other fluid.

The NTSA publication refers to Moore et al for the method used for identifying and quantitating delta-9-THC (Moore C et al (2007) Simultaneous identification of 2-carboxytetrahydrocannabinol, tetrahydrocannabinol, cannabinol and cannabidiol in oral fluid. Journal of Chromatography B: Biomedical Sciences and Applications, 852, 459-464).

Serving Limitations

The present disclosure provides servings that are below those set forth, for example, by one or more of the Washington State Legislature, Oregon State Legislature, and Colorado State Legislature.

Washington State Legislature provides: WAC 314-55-095. Marijuana servings and transaction limitations. (1) For persons age twenty-one and older and qualifying patients or designated providers who are not entered into the medical marijuana authorization database, marijuana serving and transaction limitations are as follows: (a) Single serving. A single serving of a marijuana-infused product must not exceed ten milligrams active tetrahydrocannabinol (THC), or Delta 9. (b) Maximum number of servings. The maximum number of servings in any one single unit of marijuana-infused product meant to be eaten or swallowed is ten servings or one hundred milligrams of active THC, or Delta 9. A single unit of marijuana concentrate cannot exceed one gram. RCW 69.50.101 (2rr) “THC concentration” means percent of delta-9 tetrahydrocannabinol content per dry weight of any part of the plant Cannabis, or per volume or weight of marijuana product, or the combined percent of delta-9 tetrahydrocannabinol and tetrahydrocannabinolic acid in any part of the plant Cannabis regardless of moisture content. Blood limits for DUI are defined in terms of “THC concentration”: RCW 46.20.308 (5) If after arrest and after any other applicable conditions and requirements of this section have been satisfied, a test or tests of the person's blood or breath is administered and the test results indicate that the alcohol concentration of the person's breath or blood is 0.08 or more, or the THC concentration of the person's blood is 5.00 or more, if the person is age twenty-one or over, or that the alcohol concentration of the person's breath or blood is 0.02 or more, or the THC concentration of the person's blood is above 0.00, if the person is under the age of twenty-one, or the person refuses to submit to a test, the arresting officer or other law enforcement officer at whose direction any test has been given, or the department, where applicable, if the arrest results in a test of the person's blood, shall . . . .” The present disclosure provides compositions, servings, methods of administering, methods of manufacturing, and such, that are at or below the limits set forth above.

Oregon State Legislature provides: OREGON. OAR 333-007-0310. Definitions—(20) “Delta-9 THC” is the principal psychoactive constituent (the principal cannabinoid) of cannabis, Chemical Abstracts Service Number 1972-08-3. (53) “THC” means tetrahydrocannabinol and has the same Chemical Abstracts Service Number as delta-9 THC. OAR 333-007-0210. “Maximum amount of THC” per serving of marijuana edibles=5 mg and per container=50 mg. Oregon does not have blood concentration limit of THC for purposes of assessing DUI.” The present disclosure provides compositions, servings, methods of administering, methods of manufacturing, and such, that are at or below the limits set forth above.

Colorado State Legislature provides: COLORADO. R 103—Definitions “Single-Serving Edible Retail Marijuana Product” means an Edible Retail Marijuana Product unit for sale to consumers containing no more than 10 mg of active THC. “Standardized Serving Of Marijuana” means a standardized single serving of active THC. The size of a Standardized Serving Of Marijuana shall be no more than 10 mg of active THC. “THC” means tetrahydrocannabinol. “Active THC” is not defined in statute or rule. R 602 (C). THC Content Container Restriction. Each individually packaged Edible Retail Marijuana Product, even if comprised of multiple servings, may include no more than a total of 100 milligrams of active THC. See Rule R 1004—Labeling Requirements: Specific Requirements, Edible Retail Marijuana Product. R 604 (C3). The size of a Standardized Serving Of Marijuana shall be no more than 10 mg of active THC. A Retail Marijuana Products Manufacturing Facility that manufactures Edible Retail Marijuana Product shall determine the total number of Standardized Servings Of Marijuana for each product that it manufactures. No individual Edible Retail Marijuana Product unit for sale shall contain more than 100 milligrams of active THC. CRS 42-4-1301 6(IV)—DUI Limit of 5 ng/mL blood of delta-9 THC. (IV) If at such time the driver's blood contained five nanograms or more of delta 9-tetrahydrocannabinol per milliliter in whole blood, as shown by analysis of the defendant's blood, such fact gives rise to a permissible inference that the defendant was under the influence of one or more drugs.” The internet publication, Colorado. Official State Web Portal. Colorado Marijuana (2017), provides a definition of serving, in its recitation that, “every single standardized serving (a serving consists of 10 mg of THC) of an edible retail marijuana product must be individually marked, stamped, or imprinted with the new universal symbol.”

The present disclosure provides compositions, servings, methods of administering, methods of manufacturing, and such, that are at or below the limits set forth above.

Cytochrome P450 Modulators

To provide background information, drugs can be converted in the body to inactive forms in the body by way of cytochrome P450. Cytochrome P450 is often abbreviated as “CYP,” “CYP enzymes,” or as “CYP isozymes.” The CYP enzymes occur as various isoforms and they are encoded by different genes. Each CYP isozyme acts on a specific group of substrates, and what is available are substrates that are specifically recognized by only one of the CYP enzymes. Some of these CYP isozymes and their probe substrates are shown here: Caffeine (CYP1A2); Losartan(CYP2C9); Omeprazole (CYP2C19); Dextromethorphan (CYP2D6); Midazolam (CYP3A); Bupropion (CYP2B6); Tolbutamide (CYP2C9); Chlorzoxazone (CYP2E1) (See, Grangeon A et al. (2017) J. Chromatogr. B. Analyt. Technol. Biomed. Life Sci. 1040:144-158; Snyder B D et al (2014) Eur. J. Clin. Pharmacol. 70:1115-1122; Rowland A et al (2016) Frontiers in Pharmacology. 7:517-525; Tran et al (2016) Br. J. Clin. Pharmacol. 82:160-167).

Regarding cannabinoids, CYP2C9 catalyzes the 11-hydroxylation of cannabinoids by human hepatic enzymes. Thus, the present disclosure provides inducers of CYP2C9 where administering the CYP2C9-inducer increases the 11-hydroxylation of a co-administered cannabinoid such as delta-8-THC or delta-9-THC, or a derivative thereof. For this embodiment of the present disclosure, an exemplary sequence of events is shown below. This sequence of events many involve CYP enzyme of the liver, of the gut, or of both the liver and gut:

Step One.

Administer CYP2C9 inducer, where result is increased activity of CYP3C9 in the liver.

Step Two.

Administer delta-8-THC, delta-9-THC, or a mixture of delta-8-THC and delta-9-THC.

Step Three.

The consequence is increased conversion in the liver of the administered cannabinoid to the 11-hydroxy derivative.

Regarding CYP enzyme inducers, Lumacaftor has been identified as an inducer of CYP2C9 (See, Lumacaftor/ivacaftor combination (cystic fibrosis for patients age 12 years or older with F508del mutation in CFTR gene) NDA 206-038. Page 24 of 81 page Clinical Review. 99 page Medical Review. Dabrafenib (melanoma with BRAF V600E mutation) NDA 202-806. Page 17 of 39 page Cross Discipline Team Leader Review). Also, dabrafenib has been identified as an inducer of CYP2C9 (see, Dabrafenib (melanoma with BRAF V600E mutation) NDA 202-806. Page 17 of 39 page Cross Discipline Team Leader Review). The above documents are from FDA's website, and these can be accessed by typing the name of the drug or the NDA number.

Further regarding cannabinoids, CYP3A4 catalyzes the conversion of delta-8-THC to the 7-hydroxy derivative of delta-8-THC thus reducing the concentrations of delta-8-THC in the liver. A consequence of reduced delta-8-THC in the liver is reduced conversion of delta-8-THC to 11-hydroxy-delta-8-THC. Thus, the present disclosure provides compositions and methods for increasing 11-hydroxy-delta-8-THC by coadministering CYP3A4 inhibitor with delta-8-THC to a human subject. The CYP3A4 inhibitor can be an inhibitor that is not a CYP3A4 substrate, or it can be a CYP3A4 inhibitor that is a CYP3A4 substrate where inhibition is by competitive inhibition. See, Watanabe, Yamaori, Funahashi (2007) Life Sciences. 80:1415-1419).

The present disclosure provides compositions and methods for inhibiting CYP3A4, with the consequent reduction in destruction of delta-8-THC occurring by way of CYP3A4-mediated catalysis of delta-8-THC to 7-hydroxy-delta-8-THC. This is summarized by these steps:

Step One.

Administer CYP3A4 inhibitor. CYP3A4 inhibitors include grapefruit juice, bergamottin, dihydroxybergamottin, ketoconazole, itraconazole, clarithromycine, erythromycin, atanavir, and ritonavir (see, Package label. STIVARGA (regorafenib) tablets, oral. September 2012 (15 pages). See also, Cabozantinib (thyroid cancer) NDA 203-756. Pages 34-35 of 106 page Clinical Pharmacology Review, from FDA website). Bergamottin and dihydroxybergamottin are the chemicals in grapefruit juice that inhibit CYP3A4, where the result is increased plasma levels of any drug that is normally catabolized by CYP3A4 (see, Lin H L et al (2012) Drug Metab. Dispos. 40:998-1006; He K et al (1998) Chem. Res. Toxicol. 11:252-259).

Step Two.

Administer delta-8-THC or some other THC compound that is a substrate of CYP3A4.

Step Three.

The consequence is increased concentrations of any administered delta-8-THC in the liver, where this increase results from the blocked conversion in the liver of the administered cannabinoid to the 7-hydroxy derivative.

Sesquiterpenes and Curcuminoids as Inhibitors of CYP3A4, CYP2C9, and CYP1A2

Additional CYP enzyme inhibitors are as follows. Ten sesquiterpenes (1-10) and two curcuminoids (11 and 12) were isolated from Curcuma aromatica Salisb and identified. The sesquiterpene (4S,5S)-(−)-germacrone-4,5-epoxide (7) inhibited certain subtypes of CYP more potently than or at levels comparable to the curcuminoids curcumin (11) and demethoxycurcumin (12); 7 (IC(50)=1.0±0.2 μM)>12 (IC(50)=7.0±1.7 μM)>11 (IC(50)=14.9±1.4 μM) for CYP3A4 inhibition; 12 (IC(50)=1.4±0.2 μM)>11 (IC(50)=6.0±1.4 μM)>7 (IC(50)=7.6±2.5 μM) for CYP2C9 inhibition; and 7 (IC(50)=33.2±3.6 μM) 12 (IC(50)=34.0±14.2 μM)>11 (IC(50)>100 μM) for CYP1A2 inhibition. The inhibitor compounds of greatest interest were sesquiterpene 7 and curcuminoids 11 and 12 (Bamba et al (2011) Natural Medicines. 65:583-587).

The present disclosure provides compositions and methods for co-administering delta-8-THC with one or more of sesquiterpene 7, curcuminoid 11, and curcuminoid 12. Also, the present disclosure provides compositions and methods for co-administering delta-9-THC with one or more of sesquiterpene 7, curcuminoid 11, and curcuminoid 12. The co-administering can take the form of a powder, pill, tablet, slurry, or liquid composition were the THC compound and the sesquiterpene (or curcuminoid compound) are mixed together. Also, the co-administering can take the form of a plurality of different powders, pills, tablets, slurries, or liquid compositions were the THC compound and the sesquiterpene (or curcuminoid compound) are not mixed together.

UDP-Glucuronosyltransferase (UGT) Modulators

UDP-glucouronosyltransferase (UGT) enzymes catalyze the attachment of a glucuronic acid moiety to various drugs. This conjugation promotes their excretion. UGT enzymes can catalyze attachment of a glucuronic acid moiety to the hydroxyl, carboxyl, amino, or sulfhydryl group of a target compound (See, Fujiwara R et al (2016) Structure and protein-protein interactions of human UDP-glucuronosyltransferases. Front. Pharmacol. eCollection 2016).

The present disclosure provides inhibitors of UGT enzymes that prevent conjugation of glucuronic acid to delta-8-THC or to 11-hydroxy-delta-8-THC, or that prevent conjugation of glucuronic acid to delta-9-THC or to 11-hydroxy-delta-9-THC, where preventing conjugation results in an increase in concentration of these cannabinoids in the human body. The inhibitors can be substrates of UGT enzymes (competitive inhibition). For example, 11-hydroxy-delta-9-THC is a substrate of UGT1A1 and is also a substrate of UGT1A9 (see, Mazur, Lichti, Prather (2009) Drug Metabolism Disposition. 37:1496-1504). Canagliflozin (CNF) and dapagliflozin (DPF) each can inhibit UGT1A1 and UGT1A9 (Pattanawongsa et al (2015) Drug Metab. Disposition. 43:1468-1476). Bilirubin inhibits UGTlA1 and carvacrol inhibits UGTA9 (Zeng, Shi, Zhao et al (2016) PLOS ONE. DOI:10.1371 (21 pages)). Mefenamic acid inhibits UGTA9 (Kasichayanula, Liu, Griffin et al (2012) Diabetes, Obesity and Metabolism. 15:280-283). The present disclosure provides compositions and methods for enhancing the concentration of a cannabinoid in the body, where the cannabinoid can be delta-8-THC, delta-9-THC, 11-hydroxy-delta-8-THC, 11-hydroxy-delta-9-THC, and related derivatives. The compositions and methods use one or more inhibitors of a UGT enzyme, such as canagliflozin or dapagliflozin. This embodiment can be described by these steps:

Step One.

Administer UGT enzyme inhibitor or UGT enzyme substrate.

UGT1A9 inhibitors include ginkgo flavonoids, quercitin, and kaempferol (see, Mohamed and Frye (2010) Drug Metab. Dispos. 38:270-275). UGT1A9 substrates, which may function in vivo to reduce UGT1A9-mediated glucuronidation of THC, include scopoletin, 4-methylunbelliferone, anthraflavic acid, 7-hydroxyflavone, naringenin, and 5,7-dihydroxyflavone (Albert et al (1999) Endocrinol. 140:3292-3302; Mohamed and Frye (2010) Drug Metab. Dispos. 38:270-275). UGT1A1 inhibitors include valerian, cranberry (quercetin), echinacea, and grape seed (resveratrol). UGT1A9 inhibitors include cranberry (quercetin), Ginkgo biloba, and UGT1A9 substrates include grape seed (resveratrol), and ginkgo flavonoids (Mohamed and Frye (2011) Planta Med. 77:311-321). The substrates are expected to reduce UGT-mediated conjugation of cannabinoids.

Step Two.

Administer delta-8-THC or some other THC compound that is a substrate of the same UGT enzyme.

Step Three.

The consequence is increased concentrations of any administered delta-8-THC in the liver, where this increase results from the blocked glucuronidation of the administered cannabinoid.

Co-Administering Embodiments

Without implying any limitation, “co-administering” encompasses oral administration of two different compounds, that is, as two different powders, two different pills, two different tablets, two different slurries, or two different liquids, at the same time, or in a time-frame separated by under five hours, or in a time-frame separated by under one hour, or in a time-frame separated by under ten minutes. Alternatively, “co-administering” can take the form of administering a first composition and a second composition, where the first composition does not have the same formulation as the second composition (here, the first formulation can be a powder and the second can be a pill, or the first formulation can be a slurry and the second can bea tablet, and so on).

“Co-administered” can also encompass any co-administering where the first compound has a first Cmax (ng/mL or micromolar) in the blood plasma, where the second compound has a second Cmax in the blood plasma (ng/mL or micromolar). For any chemical or compound that is absorbed by the gut, it can be expected that the compound will have a Cmax (occurring at a time defined as tmax), and that the compound will also have a C(10% max), a C(20% max), a C(50% max), and so on. C(10% max) is defined as a time occurring after tmax, where the blood concentration is ten percent that of Cmax. Using this definition, “co-administering” can be defined as an oral dosing scheme where the first compound's concentration in the bloodstream and the second compound's concentration in the bloodstream are such that C(≥10% max) for the first compound occurs coincidentally with C(≥0.10 max) for the second compound. Please notice the symbols for “greater or equal to” (≥).

“Co-administration” can also encompass administration of a first compound and of a second compound, where there is an overlap in biochemical effect. By this definition, if there is an overlap of biochemical effect, without regard to overlap of plasma concentrations of the first compound and the second compound, then this constitutes “co-administration.” The present disclosure encompasses co-administering delta-8-THC, or delta-9-THC, or a combination of delta-8-THC and delta-9-THC, with an inducer of CYP2C9. CYP2C9 catalyzes the 11-hydroxylation of THC (Watanabe et al (2007) Life Sciences. 80:1415-1419; Sachse-Seboth et al (2009) Clin. Pharmacol. Therapeutics. 85:273-276). Inducers of CYP2C9 include hyperforin (active compound in St. Johns Wort), rifampicin, phenobarbitol, and dexamethasone (Chen et al (2004) J. Pharmacol. Exp. Therapeutics. 308:495-501).

The present disclosure provides a composition comprising delta-8-THC and St. Johns Wort, either as a single formulation or as two different formulations (one containing delta-8-THC, the other containing St. Johns Wort). Also, the present disclosure provides a composition comprising delta-9-THC and St. Johns Wort, either as a single formulation or as two different formulations (one containing delta-9-THC, the other containing St. Johns Wort). In addition, the present disclosure provides a composition comprising delta-8-THC plus delta-9-THC and St. Johns Won, either as a single formulation or as two different formulations (one containing delta-8-THC plus delta-9-THC, the other containing St. Johns Wort).

Carvacrol, Curcumin, Triterpenoid Saponins, and Other Natural Products

“Carvacrol is a monoterpenic phenol produced by . . . aromatic plants, including thyme and oregano. Presently, carvacrol is used in low concentrations as a food flavoring ingredient” (Suntres, Coccimiglio, and Alipour (2015) Bio activity and Toxicological Actions of Carvacrol. Crit. Revs. Food Science Nutrition. 55:304-318). Carvacrol inhibits UGT1A9, where carvacrol inhibited the activity of 4-methylumbelliferone (the test substrate) glucuronidation, where activity was reducted to 20% maximal activity at 200 micromolar carvacrol (Dong et al (2012) Phytother. Res. 26:86-90). The role of UGT1A9 and also UGT1A10 in depleting pharmacologically active cannabinoids in the body was shown by Mazur et al (2009) Drug Metab. Dispos. 37:1496-1504, which states that, “oxidation of delta-9-THC to THC-OH results in UGT1A9 and UGT1A10 activity toward the cannabinoid.” FIG. 2 and FIG. 6A of Mazur, supra, show that THC-OH is a substrate for UGT1A9 and UGT1A10. THC-OH is “11-hydroxy-delta-9-THC.” Another publication states that, “CBN and 11-OH-THC are primarily metabolized by the extrahepatic isoform, UGT1A10, with Km values of 55 and 16 micromolar, respectively” (Radominska-Pandya et al (2008) Human hepatic and extrahepatic UDP-glucuronosyl-transferase (UGTs) enzymes involved in the metabolism of cannabinoids. FASEB J. 22 (Suppl. 711.4).

Regarding curcumin, “curcumin . . . has no known toxicities even when administered as 2% of the rat diet . . . our . . . evidence indicates that it inhibits a . . . phosphorylation requirement of UGT” (Basu, Ciotti, Hwang (2004) J. Biol. Chem. 279:1429-1441). Further regarding curcumin, “The parallel loss and recovery of both activity and phosphoserine content for UGT1A1 following curcumin treatment indicates that the mouse isozyme like human UGTs . . . undergoes required phosphorylation” (Basu et al (2007) Biochem. Biophys. Res. Commun. 360:7-13). UGT1A10 activity also depends on phosphorylation (Basu et al (2004) J. Biol. Chem. 279:28320-28329). In short, curcumin's ability to inhibit this phosphorylation results in the inhibition of a plurality of the UGT isozymes.

Regarding triterpenoid saponins, it has been found that nor-oleanane triterpenoid saponins from Stauntonia brachycanthera inhibits UGT1A10 and UGT1A1 (Liu et al (2016) Fitoterapia. 112:56-64).

The present disclosure provides compositions and methods that inhibit glucuronidation of 11-hydroxy-delta-8-THC, of 11-hydroxy-delta-9-THC, or of both 11-hydroxy-delta-8-THC and 11-hydroxy-delta-9-THC, where the composition inhibits mainly UGT enzymes of the gut, where the composition mainly inhibits hepatic UGT enzymes, or where the composition inhibits UGT enzymes of both the gut and liver. What is provided is compositions and methods comprising carvacrol, curcumin, nor-oleanane triterpenoid saponins, or any combination thereof.

The amount administered orally for each of these natural products can be, for example, about 0.1 mg, about 0.2 mg, about 0.5 mg, about 1.0 mg, about 2 mg, about 5 mg, about 10 mg, about 50 mg, about 100 mg, about 200 mg, about 500 mg, about 1,000 mg, about 5 grams, about 10 grams, and the like, of any given natural product, of a pharmacologically acceptable natural product, of a pharmacologically acceptable derivative of a natural product, or of a pharmacologically acceptable compound that is not a natural product.

“Pharmacologically acceptable” can be in terms of lack of nausea, lack of vomiting, lack of neutropenia, lack of increased serum bilirubin, lack of increased liver enzymes in serum, and so on, following oral administration of the compound. “Derivative” encompasses compounds that are methylated, phosphorylated, sulfated, formylated, conjugated with mannose, sialic acid, glucose, fucose, and the like. Derivatives that bestow increased solubility to a cannabinoid, to a terpene, or to another natural product include, glycyl esters, dialkylglycyl esters, dimethylglycyl esters, diethyl glycyl esters, amino esters, phosphate esters, and trialkylammonium glycinate, derivatives, amino acid esters containing nitrogen heterocycles as derivatives of 4-morpholinyl acetic and butyric, and 4-(4-methylpiperazinyl) acetic and butyric acids, including hydrobromide salts.

The disclosure provides a type of THC that does not lead to positive tests on blood/urine delta-9-THC tests or field sobriety tests designed to analyze delta-9-THC metabolites. Moreover, the present disclosure provides a type of THC that is not limited by per serving/package limits on delta-9-THC. The disclosure provides a prodrug to 11-OH-delta-8 THC (when ingested). The prodrug can be delta-8-THC or, alternatively, the prodrug can be delta-8-THC that is modified by covalent binding to a chemical moiety that increases solubility of delta-8-THC in water. Preferably, the covalently bound moiety is hydrolyzable in the body, providing delta-8-THC.

Transporters

The present disclosure provides inhibitors for reducing export of cannabinoids from cells, resulting in excretion from the body. Drug transporters such as P-gycoprotein (P-gp), Breast Cancer Resistance Protein (BCRP), and Organic Anion Transporters (OAT1, OAT2, OAT3) are used, in some cases, to mediate transport of drugs into cells and, in other cases, to mediate transport of drugs out of cells. Transport out of cells can be to the blood plasma, to the bile duct for excretion from the body, or transport from renal tubule cells to the urine for excretion from the body. P-glycocoprotein (Pgp) and BCRP can transport cannabinoids out of cells to the bloodstream (see, Spiro et al (2012) PLOS ONE. 7:e35937). Accordingly, the present disclosure provides compositions and methods for inhibiting drug transporters that mediate efflux of cannabinoids from cells and, more preferrably, for inhibiting drug transporters that mediate efflux out of enterocytes to the gut lumen, for inhibiting drug transporters that mediate efflux out of hepatocytes to the bile duct (see, Fakhoury et al (2005) Drug Metab. Dispos. 33:1603-1607; Bow et al (2008) Drug Metab. Dispos. 36:198-202; Scotcher et al (2017) J. Pharmacol. Exp. Ther. 116: DOI:10.1124; Mikkaichi et al (2004) Proc. Nat. Acad. Sci. 101:3569-3574).

The present disclosure provides compositions and methods for administering one or more cannabinoids and one or more compounds that inhibit efflux of cannabinoids from cells. The one or more compounds can inhibit P-glycoprotein, BCRP, or one of the OAT transporters (OAT1, OAT2, OAT3). Inhibitors of P-gp or of BCRP include drugs such as verapamil, dexverapamil, and zosuquidar, as well as natural products such as terpenes, flavonoids, and coumarins (Abdullah, Al-Abd, El-Dine et al (2015) J. Advanced Res. 6:45-62). Terpenes that inhibit drug transporters include farnesferol A, galbanic acid, limonoids such as obacunone, diterpenes such as jatrophane and lathyrane, and sesquiterpenes such as dihydro-beta-agarofuran. Flavonoids that inhibit transporters include epigallocatechin-3-gallate, 8-prenylnaringenin, and baicalein, kaempferol (from grapes), and naringenin (from grapes). Coumarins that inhibit drug transporters include furanocoumarin. The present disclosure provides compositions and methods, as outlined by the following method:

Step one. Administer an inhibitor of a drug transporter that mediates efflux of cannabinoids from enterocytes, hepatocytes, or renal tubule cells. P-glycoprotein inhibitors include zosuquidar, valspodar, elacridar, kava-kava extracts, kavalactones, fibrates, progestins (see, Weiss et al (2006) Drug Metabolism Disposition. 34:203-207). Curcumin is a P-glycoprotein inhibitor (Neerati et al (2013) J. Cancer Sci. Ther. 5:313-319).

Step two. Administer delta-8-THC or some other THC compound that is a substrate of the same transporter.

Step three. The consequence is increased concentrations of any administered delta-8-THC in the bloodstream and also in the liver, where this increase results from the blocked efflux and consequent prevention of clearance from the body.

Identifying Compounds that Inhibit Cannabinoid Catabolism, where the Compounds can be Taken Orally

Compounds that can inhibit cannabinoid catabolism include CYP enzyme inhibitors, CYP enzymes substrates, UGT enzyme inhibitors, UGT enzyme substrates, P-gp inhibitors and P-gp substrates. Substrates of these types inhibit by way of competitive inhibition. For assays that involve assays that detect CYP enzyme activities using microsomes as the source of enzyme, Promega Corp. provides the following information on methodology. Large amounts of protein or phospholipid from microsome preparations can bind nonspecifically to a drug or inhibitor, leading to a reduction in the effective concentration and overestimation of Km and Ki values (Technical Bulletin. P450-Glo® Assays. Promega Corp., Madison, Wis.). Preparations of CYP enzymes are available from Corning, Sigma-Aldrich, Life Technologies, Xenotech, Cypex, New England Biolabs, Oxford Biomedical Research, BioreclamationIVT, and Moltox, Inc.

Corning Supersomes® take the form of microsomes engineered to contain recombinant CYP enzymes, recombinant UGT enzymes, or other drug-metabolizing enzymes of the microsomal fraction of the liver (Stresser et al (2013) Cytochrome P450 Enzyme Mapping in Drug Discovery using Corning® Supersomes® EnzymesApplication Note 467. Corning, Inc., Tewksbury, Mass.).

Regarding CYP enzymes and UDP-glucouronosyltransferase (UGT) enzymes, further information on reagents and methodology is available (see, e.g., Li et al (2015) High-throughput cytochrome P450 cocktail inhibition assay for assessing drug-drug and drug-botanical interactions. Drug Metab. Dispos. 43:1670-1678; Lee et al (2015) Simultaneous screening of activities of five cytochrome P450 and four uridine 5′-diphospho-glucuronosyltransferase enzymes in human liver microsomes using cocktail incubation and liquid chromatography tandem mass spectrometry. Drug Metab. Dispos. 43:1137-11146; Seo et al (2014) In vitro assay of six UDP-glucuronosyltransferase isoforms in human liver microsomes, using cocktails of probe substrates and liquid chromatography-tandem mass spectrometry. Drug Metab. Dispos. 42:1803-1810; Walsky et al (2012) Optimized assays for human UDP-glucuronosyltransferase (UGT) activities: altered alamethicin concentration and utility to screen for UGT inhibitors. Drug Metab. Dispos. 40:1051-1065).

Screening for terpenes that inhibit CYP enzymes (BD Biosciences assay). BD Gentest® Pooled Human Liver Microsomes takes the form of human liver microsomes that comprise many cytochrome P450 enzymes, most notably, CYP1A2, CYP2C9, CYP2C19, CYP2D6, and CYP3A4. Terpenes or other candidate compounds can be screened for their ability to inhibit CYP enzymes, as follows. The setup for the screening assay provides direct information as to the influence of terpenes on CYP enzyme-mediated catabolism of a cannabinoid of choice, such as delta-8-THC. An assay mixture can contain the Gentest® Pooled Human Liver Microsomes plus delta-8-THC plus a terpene, such as limonene.

Alternatively, the assay mixture can contain the Gentest Pooled Human Liver Microsomes plus delta-8-THC plus a cocktail of terpenes, where the cocktail takes the form of a mixture of two, three, four, five, six, or seven of the terpenes selected from alpha-bisabolol, borneol, camphene, camphor, beta-caryophyllene, delta-3-carene, caryophyllene, caryophyllene oxide, alpha-cedreen, beta-eudesmol, fenchol, geraniol, guaiol, alpha-humulene, isoborneol, limonene, linalool, menthol, myrcene, nerol, cis-ocimene, trans-ocimene, alpha-phellandrene, beta-pinene, sabinene, alpha-terpinene, alpha-terpineol, terpinolene, alpha-guaiene, elemene, farnesene, germacrene B, guaia-1(10),11-diene, trans-2-pinanol, selina-3,7(11)-diene, eudesm-7(11)-en4-ol, and valencene. Preferred terpenes are disclosed by US2015/0152018 of Raber and Elzinga, which is incorporated herein in its entirety.

The ability of terpenes to inhibit CYP enzyme-mediated catabolism of the cannabinoid can be measured by quantifying the cannabinoid following incubations plus or minus the added terpene (or plus or minus the terpene cocktail). Quantification can be with high pressure liquid chromatography (HPLC).

Screening for terpenes that inhibit CYP enzymes (Promega assay). The present disclosure provides reagents and methods for identifying compounds of interest that inhibit CYP enzymes that catabolize a cannabinoid. For example, what is provided is reagents and methods for identifying a terpene (or a cocktail of selected terpenes), that inhibit CYP enzyme mediated catabolism of delta-8-THC. The P450-Glo® Assays described below can identify terpenes that inhibit CYP enzymes, where this assay uses a standard substrate (the substrate is not a cannabinoid; it is provided by Promega). After identifying terpenes of interest using the convenient P450-Glo® Assays, where Promega's substrate is used, assays using isolated human liver microsomes or using isolated CYP enzymes can be used. Here, the experimental setup is to test the inhibitory effect of the terpenes of interest where the substrate is delta-8-THC. The inhibitory effect is determined by HPLC analysis of delta-8-THC from incubations plus or minus the terpene.

P450-Glo® Assays provide a luminescent method to measure CYP enzyme activity. The assays test the effects of drugs or other compounds on CYP enzyme activities. All of these assays can be used for cell-free CYP inhibition studies. Many of these assays also can be used for cell-based CYP induction assays. Promega provides P450-Glo® Substrates. These are CYP enzyme substrates that are proluciferins derivatives of beetle luciferin [(4S)-4,5-dihydro-2-(6′-hydroxy-2′-benzothiazolyl)-4-thiazolecarboxylic acid]. The derivatives are converted by CYP enzymes to luciferin products. d-Luciferin is formed and detected in a second reaction with Promega's Luciferin Detection Reagent. The amount of light produced in the second reaction is proportional to CYP activity (Promega Corp, Madison, Wis.).

Embodiments that Inhibit Conversion of 11-Hydroxyl-Delta-8-THC or of 11-Hydroxy-Delta-9-THC to 1-Nor-8-Carboxy-THC

The present disclosure provides compositions that inhibit the conversion of 11-hydroxy-delta-8-THC or of 11-hydroxy-delta-9-THC to the inactive 11-nor-8-carboxy-THC compound. This embodiment is based on the, “assumption that 11-hydroxyTHC is oxidized to the carboxaldehyde by alcohol dehydrogenases, and further oxidation to the carboxylic acid catalyzed by aldehyde dehydrogenases or aldehyde oxidases” (Dr. Patrick Callery, email of Aug. 15, 2017).

Assessing if a Terpene or Other Compound is a CYP Enzyme Inhibitor

The following table from BD Biosciences discloses standard compounds that are standard CYP enzyme inhibitors and CYP enzyme substrates. Where a terpene of the present disclosure is found to have a Km that is similar to that of one of the CYP enzyme substrates, or where a terpene of the present disclosure is found to have a Ki that is similar to that of one of the CYP enzyme inhibitors, then the terpene can be considered to be an inhibitor. Also, where a terpene of the present disclosure is found to have a Km below (or far below) that of one of the CYP enzyme substrates, or where a terpene of the present disclosure is found to have a Ki that is below (or far below) that of one of the CYP enzyme inhibitors, then the terpene can be considered to be an inhibitor. The above statements are with regard to the situation where the CYP enzyme substrate is a cannabinoid, such as delta-8-THC. Where the terpene is a substrate, it may be a competitive inhibitor. Where the terpene is an inhibitor but not a substrate, and where it inhibits, then it may be a direct inhibitor.

TABLE 1
CYP ISOZYMES
Table 1. CYP isozymes. Standard inhibitors and standard substrates

| Cytochrome P450 | | | |
|---|---|---|---|
| isozyme | Inhibitor | Substrate and Km micromolar | |
| CYP1A2 | furafyline | phenacetin | 24 |
| CYP2A6 | tranylcypromine | coumarin | 1.0 |
| CYP2B6 | ketoconazole | bupropion | 137 |
| CYP2C8 | montelukast | amodiaquine | 0.9 |
| CYP2C9 | sulfaphenazole | diclofenac | 3.5 |
| | tienilic acid | | |
| CYP2C19 | S(+)N(3) | S-mephenytoin | 24 |
| | benzylnirivanol | | |
| CYP2D6 | quinidine | dextromorphone | 5.0 |
| CYP2E1 | chlormethiazole, | chlorzoxazone | 68 |
| | disulfiram | | |
| CYP3A4 | ketoconazole | midazolam | 1.8 |
| | azamulin | | |
| CYP3A4 | ketoconazole | testosterone | 64 |
| | azamulin | | |
| CYP3A4 | ketoconazole | nifedipine | — |
| | azamulin | | |

BD Biosciences (2010) BD Tissue Fractions. Reagents for Drug Metabolism. BD Biosciences. Bedford, MA (16 pages)

Cannabinoids

One of more of the following cannabinoids can be included in the compositions of the present disclosure. Alternatively, one of more of the following cannabinoids can be excluded (omitted) from the compositions and methods of the present disclosure. Cannaboids and related compounds include, for example, cannabigerol; cannabichromene; cannabitriol; cannabidiol; cannabicyclolol; cannabielsoin, cannabinodiol; cannabinol; delta-8-tetrahydrocannabinol; delta-9-tetrahydrocannabinol; cannabichromanone; cannabicoumaronone; cannabicitran; 10-oxo-delta-6a10a-tetrahydrocannabinol; cannabiglendol; delta-7-isotetmbydrocannabinol; CBLVA; CBV; CBEVA-B; CBCVA; delta-9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisoside; vitexin; canniprene; 3,4′-dihydroxy-5-methoxy bibenzyl; dihydroresveratrol; 3,4′-dihydroxy-5,3′-dimethoxy-5′-isoprenyl; cannabistilbene 1; cannabistilbene 11a; cannabistilbene 11b; cannithrene 1; cannithrene 2; cannabispirone; iso-cannabispirone; cannabispirenon-A; cannabispirenone-B; cannabispiradienone; alpha-cannabispiranol; beta-cannabispiranol; acetyl-cannabispirol; 7-hydroxy-5-methoxyindan-1-spiro-cyclohexane; 5-hydroxy-7-methoxyindan-1-spiro cyclohexane; myristic acid, palmitic acid, oleic acid, stearic acid, linoleic acid, linolenic acid, arachidic acid, eicosenoic acid, behenic acid, lignoceric acid, 5,7-dihydroxyindan-1-cyclohexane; cannabispiradienone; 3,4′-dihydroxy-5-methoxybibenzyl; canniprene; cannabispirone; cannithrene 1; cannithrene 2; alpha-cannabispiranol; acetyl-cannabispirol; vomifoliol; dihydrovomifoliol; beta-ionone; dihydroactinidiolide; palustrine; palustridine; plus-cannabisativine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary metabolism in cannabis in Phytochem. Rev. DOI 10.1007/s11101-008-9094-4).

Measuring Cannabinoids

Cannabinoids can be separated, purified, analyzed, and quantified by a number of techniques. Available equipment and methods include, e.g., gas chromatography, HPLC (high pressure liquid chromatography, high performance liquid chromatography), mass spectrometry, time-of-flight mass spectrometry, gas chromatography-mass spectrometry (GC-MS), and liquid chromatography-mass spectrometry (LC-MS). Equipment for separation and analysis is available from Waters Corp., Milford, Mass.; Agilent, Foster City, Calif.; Applied Biosystems, Foster City, Calif.; and Bio-Rad Corp., Hercules, Calif.

The present disclosure provides in-line monitoring of purification, that is, quantitation of THC as well as quantitation of impurities. In-line monitoring may be by UPLC methods, or by other methods. Ultra-high performance liquid chromatography (UPLC) is similar to HPLC, except that UPLC uses smaller particles in the column bed, and greater pressures. The particles can be under 2 micrometers in diameter, and pressures can be nearly 15,000 psi. UPLC also uses higher flow rates, and can provide superior resolution and run times in the range of under 30 seconds (Wren and Tchelitcheff (2006) J. Chromatography A. 1119:140-146; Swartz, M. E. (May 2005) Separation Science Redefined). The application of UPLC to cannabinoids has been described (see, Jamey et al (2008) J. Analytical Toxicology. 32:349-354; Badawi et al (2009) Clinical Chemistry. 55:2004-2018). Suitable UPLC columns for cannabinoid analysis include, e.g., Acquity®UPLC HSS T3 C18, and Acquity® UPLC BEH C18 column (Waters, Milford, Mass.). Other methods for detecting cannabinoids include, e.g., infrared (IR) spectroscopy, gas chromatography mass spectroscopy (GCMS), and electrospray tandem mass spectroscopy (ESI-MS/MS)(Ernst et al (2012) Forensic Sci. Int. 222:216-222).

Various Numbering Systems for Cannabinoids

The present disclosure uses the nomenclature as set forth by Pertwee R G et al (2010) International Union of Basic and Clinical Pharmacology. LXXIX. Cannabinoid receptors and their ligands: beyond CB1 and CB1. Pharmacol. Rev. 62:588-631. Regarding different numbering systems for the same compound, AVIV (US 2004/0110827) states that: “It should be noted that for historical reasons, these cannabinoid analogs are still named following the previous nomenclature, where the terpenic ring was the base for the numbering system. Then the chiral centers of THC type cannabinoids were at carbon atoms 3 and 4. The accepted nomenclature is now based on the phenolic ring as the starting point for numbering. Thus, THC that was previously described as delta-1-THC was later renamed delta-9-THC, similarly delta-6-THC was renamed delta-8-THC, and the chiral centers are at carbons 6a and 10a.” AVIV also has this comment about enantiomers: “delta-9-THC was established by Mechoulam R. et al. in 1967 and found to be of (−)-(3R,4R) stereochemistry. It was later found that the psychotropic activity of cannabinoids resides in the natural (3R,4R) OH series, while the opposite enantiomeric synthetic series (3S,4S) was free of these undesirable effects.”

According to Chulgin, the numbering system most broadly used recognizes both the terpene nature and the aromatic nature of the two different parts of the cannabinoid. Here, the terpene is numbered from the ringcarbon that carries that branched methyl group, and this is numbered 7, and the remaining three carbons of the isopropyl group are then numbered sequentially. The advantage to this numbering system is that this numbering system is applicable whether the center ring is closed or open. Other numbering systems are the biphenyl numbering system, the Chemical Abstracts system (substituted dibenzopyran numbering), and the Todd numbering system (pyran numbering) (see, Chulgin AT (1969) Recent developments in cannabis chemistry. J. Psychedelic Drugs. pp. 397-415.

Terpenes

The present disclosure provides terpenes, either endogenous or exogenous (intentionally added), as a component of a cannabinoid composition. Biochemical properties of terpenes, including receptor binding, can be assessed using labeled terpenes and labeled ligands where a terpene influences binding properties of the labeled ligand. Useful labels include radioactive labels, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

Terpenes modify and modulate the effects of THC and other cannabinoids and impact the overall medicinal properties of the particular cultivar. Physiological effects can be detected when inhaled from ambient air, where the result is serum levels in the single digit ng/mL range (see, US 2015/0080265 of Elzinga and Raber, which is incorporated herein by reference in its entirety). Terpenes display unique therapeutic effects that may contribute to the overall effects of medicinal cannabis. The synergy of terpenes and cannabinoids are likely responsible for providing the effective treatment of pain, anxiety, epilepsy, inflammation, depression, and infections (McPartland and Russo (2001) J. Cannabis Ther. 1:103-132).

The term “entourage effect” refers to the influence of the combination of cannabinoids and terpenes that results in synergic effects on physiology (Russo (2011) Brit. J. Pharmacol. 163:1344-1364; Cornal (2001) J. Cannabis Therapeutics. vol. 1, issue 3-4). Terpenes in cannabis have been described. See, Flores-Sanchez and Verpoorte (2008) Phytochem. Rev. 7:615-639, and US2015/0080265 of Elzinga and Raber and US2015/0152018 of Raber and Elzinga, each of which is incorporated herein in its entirety.

Dose Embodiments

A dose for oral administration, in embodiments, contains about 0.1 mg prodrug, about 0.2 mg prodrug, about 0.3 mg prodrug, about 0.4 mg prodrug, about 0.5 mg prodrug, about 1.0 mg prodrug, about 2.0 mg prodrug, about 3.0 mg prodrug, about 4.0 mg prodrug, about 5.0 mg prodrug, about 6.0 mg prodrug, about 7.0 mg prodrug, about 8.0 mg prodrug, about 9.0 mg prodrug, about 10 mg prodrug, about 20 mg prodrug, about 30 mg prodrug, about 40 mg prodrug, about 50 mg prodrug, about 60 mg prodrug, about 70 mg prodrug, about 80 mg prodrug, about 90 mg prodrug, about 100 mg prodrug, about 150 mg prodrug, about 200 mg prodrug, about 250 mg prodrug, about 300 mg prodrug, about 350 mg prodrug, about 400 mg prodrug, about 500 mg prodrug, and the like. Also provided is any range consisting of a combination of any two of these quantities.

In exclusionary embodiments, what can be excluded is any oral dose that provides less than any of these quantities, or that provides more than any of these quantities.

Also provided is a dose for oral administration, in embodiments, that contains 0.1-0.5 mg prodrug, 0.5-1.0 mg prodrug, 2.0-5.0 mg prodrug, 5.0-10.0 mg prodrug, 10-20 mg prodrug, 20-50 mg prodrug, 50-100 mg prodrug, 100-200 mg prodrug, 200-500 mg prodrug, 500-1000 mg prodrug, and the like, or any range consisting of a combination or sum of any or all of these ranges. In exclusionary embodiments, what can be excluded is any oral dose that provides less than any of these quantities, or that provides more than any of these quantities.

Delta-8-THC/Delta-9-THC Ratio Embodiments

This provides ranges that are in low amounts, where the disclosure provides an orally acceptable composition that is orally acceptable to the human subject, and where the composition provides in a weight/weight ratio [delta-8-THC]/[delta-9-THC] of: 5 mg/2.5 mg, 5 mg/2.0 mg, 5 mg/1.5 mg, 5 mg/1.25 mg, 5 mg/1.0 mg, 5 mg/0.75 mg, 5 mg/0.5 mg, 5 mg/0.25 mg. Also provided is, 2.5 mg/2.5 mg, 2.5 mg/2.0 mg, 2.5 mg/1.5 mg, 2.5 mg/1.25 mg, 2.5 mg/1.0 mg, 2.5 mg/0.75 mg, 2.5 mg/0.5 mg, 2.5 mg/0.25 mg.

Also encompassed are “about” embodiments, where each of the recited ratios is preceded by the term “about.” Further encompassed are exclusionary embodiments, where each of the recited ratios is preceded by the phrase, “wherein what is excluded is compositions with the weight/weight ratio of” or “wherein what is excluded is compositions with the weight/weight ratio of about.”

Further ranges that use low amounts include, weight/weight ratio [delta-8-THC]/[delta-9-THC] of: 2 mg/2.5 mg, 2 mg/2.0 mg, 2 mg/1.5 mg, 2 mg/1.25 mg, 2 mg/1.0 mg, 2 mg/0.75 mg, 2 mg/0.5 mg, 2 mg/0.25 mg. Also provided is, 1.5 mg/2.5 mg, 1.5 mg/2.0 mg, 1.5 mg/1.5 mg, 1.5 mg/1.25 mg, 1.5 mg/1.0 mg, 1.5 mg/0.75 mg, 1.5 mg/0.5 mg, 1.5 mg/0.25 mg. Further provided is weight/weight ratio [delta-8-THC]/[delta-9-THC] of: 1.0 mg/2.5 mg, 1.0 mg/2.0 mg, 1.0 mg/1.5 mg, 1.0 mg/1.25 mg, 1.0 mg/1.0 mg, 1.0 mg/0.75 mg, 1.0 mg/0.5 mg, 1.0 mg/0.25 mg. Additionally provided is weight/weight ratio [delta-8-THC]/[delta-9-THC] of: 0.5 mg/2.5 mg, 0.5 mg/2.0 mg, 0.5 mg/1.5 mg, 0.5 mg/1.25 mg, 0.5 mg/1.0 mg, 0.5 mg/0.75 mg, 0.5 mg/0.5 mg, 0.5 mg/0.25 mg.

Also encompassed are “about” embodiments, where each of the recited ratios is preceded by the term “about.” Further encompassed are exclusionary embodiments, where each of the recited ratios is preceded by the phrase, “wherein what is excluded is compositions with the weight/weight ratio of” or “wherein what is excluded is compositions with the weight/weight ratio of about.”

The present disclosure provides an orally acceptable composition, that is orally acceptable to a human subject, and where the composition provides in a weight/weight ratio [delta-8-THC]/[delta-9-THC] of: 5 mg/5 mg, 10 mg/5 mg, 15 mg/5 mg, 20 mg/5 mg, 25 mg/5 mg, 30 mg/5 mg, 35 mg/5 mg, 40 mg/5 mg, 45 mg/5 mg, 50 mg/5 mg, 60 mg/5 mg, 70 mg/5 mg, 80 mg/5 mg, 90 mg/5 mg, 100 mg/5 mg, 120 mg/5 mg, 140 mg/5 mg, 150 mg/5 mg, 160 mg/5 mg, 180 mg/5 mg, 200 mg/5 mg, and the like.

Also encompassed are “about” embodiments, where each of the recited ratios is preceded by the term “about.” Further encompassed are exclusionary embodiments, where each of the recited ratios is preceded by the phrase, “wherein what is excluded is compositions with the weight/weight ratio of” or “wherein what is excluded is compositions with the weight/weight ratio of about.”

This provides ranges in greater amounts than disclosed above. The present disclosure provides an orally acceptable composition, that is orally acceptable to a human subject, and where the composition provides in a weight/weight ratio [delta-8-THC]/[delta-9-THC] of: 5 mg/10 mg, 10 mg/10 mg, 15 mg/10 mg, 20 mg/10 mg, 25 mg/10 mg, 30 mg/10 mg, 35 mg/10 mg, 40 mg/10 mg, 45 mg/10 mg, 50 mg/10 mg, 60 mg/10 mg, 70 mg/10 mg, 80 mg/10 mg, 90 mg/10 mg, 100 mg/10 mg, 120 mg/10 mg, 140 mg/10 mg, 150 mg/10 mg, 160 mg/10 mg, 180 mg/10 mg, 200 mg/10 mg, and the like.

Also encompassed are “about” embodiments, where each of the recited ratios is preceded by the term “about.” Further encompassed are exclusionary embodiments, where each of the recited ratios is preceded by the phrase, “wherein what is excluded is compositions with the weight/weight ratio of” or “wherein what is excluded is compositions with the weight/weight ratio of about.”

This provides even greater amounts for the ranges. The present disclosure provides an orally acceptable composition, that is orally acceptable to a human subject, and where the composition provides in a weight/weight ratio [delta-8-THC]i[delta-9-THC] of: 5 mg/2 ng, 10 mg/20 mg, 15 mg/20 mg, 20 mg/20 mg, 25 mg/20 mg, 30 mg/20 mg, 35 mg/20 mg, 40 mg/20 mg, 45 mg/20 mg, 50 mg/20 mg, 60 mg/20 mg, 70 mg/20 mg, 80 mg/20 mg, 90 mg/20 mg, 100 mg/20 mg, 120 mg/20 mg, 140 mg/20 mg, 150 mg/20 mg, 160 mg/20 mg, 180 mg/20 mg, 200 mg/10 mg, and the like.

Also encompassed are “about” embodiments, where each of the recited ratios is preceded by the term “about.” Further encompassed are exclusionary embodiments, where each of the recited ratios is preceded by the phrase, “wherein what is excluded is compositions with the weight/weight ratio of” or “wherein what is excluded is compositions with the weight/weight ratio of about.”

Regarding compositions that do not contain any delta-9-THC, the present disclosure provides an orally acceptable composition, that is orally acceptable to a human subject, where the composition comprises delta-8-THC, or a derivative of delta-8-THC, or a combination of delta-8-THC plus a derivative of delta-8-THC, but does not include any detectable delta-9-THC. The composition can contain, for example, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1000 mg of delta-8-THC.

In “about” embodiments, the composition can contain, for example, about 0.1 mg, about about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 30 mg, about 40 mg, about 50 mg, 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, or about 1000 mg of delta-8-THC.

Regarding compositions that do not contain any delta-9-THC derivatives, the present disclosure provides an orally acceptable composition, that is orally acceptable to a human subject, where the composition comprises delta-8-THC, or a derivative of delta-8-THC, or a combination of delta-8-THC plus a derivative of delta-8-THC, but does not include any detectable delta-9-THC derivatives. In embodiments, the limit for detectability can be 1,000,000 picograms (pg), 500,000 pg, 200,000 pg, 100,000 pg, 50,000 pg, 20,000 pg, 10,000 pg, 5,000 pg, 2,000 pg, 1,000 pg, 500 pg, 200 pg, 100 pg, 50 pg, 20 pg, 10 pg, and the like. Detection can use high pressure liquid chromatography (HPLC), gas chromatograph (GC), mass spectrometry, GC-mass spec, MALDI-TOF (see, e.g., Gottardo R et al (2012) Direct screening of herbal blends for new synthetic cannabinoids by MALDI-TOF MS. J. Mass Spectrom. 47:141-146; Hall B J et at (1998) Determination of cannabinoids in water and human saliva by solid-phase microextraction and quadrupole ion trap gas chromatography/mass spectrometry. Anal. Chem. 70:1788-1798), and so on.

In embodiments, the present disclosure provides one or more doses that is oral, topical, intravenous (iv), intranasal, mucosal, intraperitoneal (ip), rectal, or any combination of routes thereof.

Prodrug Embodiments

Delta-8-THC is a suitable prodrug for conversion in the body to 1-hydroxy-delta-8-THC. The present disclosure provides prodrugs that am convertable in the human body to 11-hydroxy-delta-8-THC. Delta-8-THC has desired psychological effects on human subjects, and 11-hydroxy-delta-g-THC also has desired psychological effected on human subjects, where the effects of 11-hydroxy-delta-8-THC are greater than those of delta-8-THC. Other suitable prodrugs are derivative s of delta-8-THC that are hydroxylated, phosphorylated, methylated, acetylated, glycosylated, and so on. Also, suitable prodrugs are derivatives of delta-8-THC that contain a moiety hydrolyzable by an enzyme expressed by human cells, such as enterocytes, pancreatic exocrine cells, or hepatocytes.

Further Chemical Embodiments

The present disclosure provides compositions, and related methods, that comprise cannabinoid compounds that are not naturally produced by cannabis plants. Delta-8-THC and 11-hydroxy-delta-8-THC are not naturally produced by cannabis plants. The present disclosure provides compositions and methods that optionally can exclude in vitro (lab bench) allylic oxidation reactions of cannabinoids. Also, the disclosure can exclude, in some embodiments, compositions that do not contain a double bond in the 8-position of any cannabinoid. The present disclosure provides compositions and methods that provide a mixture of oxidative products produced in the body where, optionally, the mixture of oxidative products possesses different positions, different chiralities, or both different positions and also different chiralities. In an exclusionary embodiment, the present disclosure provides in vitro compositions and methods comprising delta-8-THC but excluding 11-hydroxy-delta-8-THC. Also, provided are in vitro compositions and methods comprising delta-8-THC and 11-hydroxy-delta-THC where the ratio of ((delta-8-THC)/(11-hydroxy-delta-8-THC)) on a molar basis is at least 1/1/, at least 211, at least 4/1, at least 8/1, at least 10/1, at least 20/1, at least 50/1, at least 100/1, at least 200/1, and the like. In one aspect, the composition is a pharmaceutical composition that it exists outside of the human body and is capable of administering to a human subject, or exists outside of the human body and outside any plant cell and is capable of administering to a human subject, or exists outside of the human body and is not in contact with any plant cell and is capable of administering to a human subject.

Receptor Binding Methods

The cannabinoid receptors include CB1 and CB2. CB1 and CB2 are members of the G protein-coupled receptor family. The ligands of CB1 include delta-9-tetrahydrocannabinol (delta-9-THC), as well as an endogenous ligand, N-arachidonyl ethanolamide (AEA; anandamide). In addition to CB1 and CB2, cannabinoids can bind to “receptors” such as various ion channels, such as vanilloid (TRPV) receptors, and to nuclear receptors, such as peroxisome proliferator-activated receptor (PPAR) (Console-Bram et al (2012) Prog. Neuropsycho-pharmacol. Biol. Psychiatry. 38:4-15; US2015/008025 of Elzinga and Raber, which is incorporated herein by reference in its entirety). Biochemical properties of terpenes, including receptor binding, can be assessed using labeled terpenes and labeled ligands where a terpene influences binding properties of the labeled ligand. Useful labels include ³²P, ³³P, ³⁵S, ¹⁴C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

A suitable background, context, and starting point for understanding CB1 and CB2 receptors is provided by the following data (Table 2) on cells expressing either human CB receptor or human CB2 receptor (Radwan et al (2015) J. Natural Products. 78:1271-1276; Hayakawa et al (2010) Pharmaceuticals. 3:2197-2212). Radioligand binding assays were performed to test binding affinity for various cannabinoid compounds. Compound 3, for example, bound tightly to CB1 and to CB2, where the binding was comparable to that of delta-8-THC or delta-9-THC. Compound 3 was a partial agonist of both receptors.

TABLE 2
Table 2. Data from Radwan et al (2015)
J. Natural Products. 78: 1271-1276.

| | | Binding | | Binding | |
|---|---|---|---|---|---|
| | | affinity | | affinity | |
| | | to CB1 | | to CB2 | |
| Compound 1 | 8-alpha-hydroxy- | 1906 | nM | 3219 | nM |
| | delta-9-THC | | | | |
| Compound 2 | 8-beta-hydroxy- | 65 | nM | 88 | nM |
| | delta-9-THC | | | | |
| Compound 3 | 10-alpha-hydroxy- | 31 | nM | 30 | nM |
| | delta-8-THC | | | | |
| Compound 4 | 10-beta-hydroxy- | 830 | nM | 3274 | nM |
| | delta-9,11-hexahydro- | | | | |
| | cannabinol | | | | |
| Compound 5 | 10-alpha-hydroxy- | 117 | nM | 129 | nM |
| | delta-9,11-hexahydro- | | | | |
| | cannabinol | | | | |
| delta-8-THC | — | 78 | nM | 12 | nM |
| delta-9-THC | — | 18 | nM | 42 | nM |

Each of delta-8-THC and 11-hydroxy-delta-8-THC are agonists to CB1. Also, each of delta-9-THC and 11-hydroxy-delta-9-THC are agonists to CB1. Corresponding information on 11-hydroxy-delta-8-THC and 11-hydroxy-delta-9-THC, as it applies to CB2, may be available.

The present disclosure provides a composition comprising a mixture of delta-8-THC and delta-9-THC, where the delta-8-THC amplifies a signal provoked by delta-9-THC. Also, what is provided is a composition comprising a mixture of delta-8-THC and delta-9-THC, where the delta-9-THC amplifies a signal provoked by delta-8-THC.

The present disclosure provides a composition comprising a mixture of a delta-8-THC derivative and delta-9-THC, where the delta-8-THC derivative amplifies a signal provoked by delta-9-THC. Also, what is provided is a composition comprising a mixture of delta-8-THC derivative and delta-9-THC, where the delta-9-THC amplifies a signal provoked by the delta-8-THC derivative.

The present disclosure provides a composition comprising a mixture of delta-8-THC and a delta-9-THC derivative, where the delta-8-THC amplifies a signal provoked by delta-9-THC derivative. Also, what is provided is a composition comprising a mixture of delta-8-THC and delta-9-THC derivative, where the delta-9-THC derivative amplifies a signal provoked by delta-8-THC.

Treatments for Human Subjects

This concerns traumatic brain injury. The present disclosure provides a pro-drug to 11-hydroxy-delta-8-THC, where administration is by oral ingestion. This is for treating or preventing traumatic brain injury, or chronic traumatic encephalopathy (CTE). Delta-8 has the double bond in the same position as dexanabinol. Oxidation of delta-8-THC by enzymes of the liver produces 11-hydroxy-delta-8-THC.

Humulus lupulus L. Cannabaceae, and extracted compounds, are useful for treating anxiety and insomnia, mild pain reduction, dyspepsia, inflammation, or liver injury (Weiskirchen et al (2015) Front Physiol. 6:140. doi: 10.3389).

Cannabis species with higher levels of CBD were shown to have greater efficacy against insomnia, and that Cannabis sativa had greater efficacy against nightmares, when compared to Cannabis indica (Belendiuk et al (2015) Addictive Behaviors. 50:178-181). Also, Cannabis indicia showed greater efficacy for improving energy and appetite, as compared with Cannabis sativa (Corral (2001) J. Cannabis Therapeutics. vol. 1, issue 3-4). Cannabis, or extracts thereof have been shown to be effective in preventing or reducing pain, sleep disturbance, and spams (see, e.g., Rog et al (2005) Neurology. 65:812-819; Wade et al (2004) Multiple Sclerosis Journal. 10:434-441).

Inhaling Embodiments

Aerosols and dry powder formulations for inhaling are available. See, Mitchell, Nagel, Wiersema, and Doyle (2003) AAPS PharmSciTech. 4(4) Article 54 (9 pages); Asai et al (2016) Pharm. Res. 33:487-497; Kopsch et al (2017) Int. J. Pharm. 529:589-596; Fishler and Sznitman (2017) Inhalation. 11:21-25. Vaporizers are available, for example, from Storz and Bickel (Tuttlingen. Germany), Arizer Tech (Waterloo, Canada), Organicex (Las Vegas, Nev.), and Elemental Technologies (Seattle, Wash.).

Psychology Embodiments and Methods

The disclosure provides compositions and methods that avoid the psychoactive effects of delta-9-THC. Reasons to avoid psychoactive effects of delta-9-THC include the fact that psychoactivity is viewed as an unwanted side-effect in typical medical treatments; and that psychoactivity is sometimes an unwanted response associated with social norms, as has been documented for drinking alcohol and intoxication (see, Robin and Johnson (1996) Attitude and peer cross pressure: adolescent drug alcohol use. J. Drug Educ. 26:69-99; Room (2009) Stigma, social inequality and alcohol and drug use. Drug Alcohol. Rev. 24:143-155). The following concerns non-psychoactive effects of delta-8-THC. Delta-8-THC has useful physiological activity mediated through CB receptors separate from psychoactivity. There is value in decoupling these two types of CB receptors. In a preferred embodiment, the present disclosure provides compositions and methods that have both: (1) Non-psychoactive effects, and (2) Psychoactive effects. To explain this preferred embodiment, the compositions of the present disclosure reduce the psychoactivity or reduce the detectability of that psychoactivity in comparison to delta-9-THC. In the case of a prodrug (for example, the prodrug being delta-8-THC), it is the case that delta-8-THC is devoid of psychoactivity but that the metabolite of delta-8-THC (the metabolite being 11-hydroxy-delta-8-THC), does possess psychoactivity.

The present disclosure provides compositions and methods with psychoactive effects that occur when delta-8-THC, or a derivative thereof, that is administered alone and then converted in the body to 11-hydroxy-delta-8-THC, where the non-psychoactive effect is one or more of: (1) Relaxation; (2) State of well-being; and (3) Decreased REM and increased deep sleep.

Also, the disclosure provides compositions and methods with non-psychoactive effects that occur when delta-8-THC, or a derivative thereof, that is administered alone and then converted in the body to 11-hydroxy-delta-8-THC, where the non-psychoactive effect is one or more of: (1) Increased restful sleep, (2) Neuroprotection, and (3) Anorexia.

(1) Increased restful sleep. Restful sleep inhuman subjects can be assessed by the Behavioral Risk Factor Surveillance System (BRFSS) sleep questions (Jungquist et al (2016) 12:1585-1592), polysomnography or gas exchange monitoring (Huttmann et al (2007) Lung. 195:361-369). Devine et al (2005) Pharmacoeconomics. 23:889-912 describe various instruments for assessing human sleep: Basic Nordic Sleep Questionnaire, Leeds Sleep Evaluation Questionnaire, Medical Outcomes Study—Sleep Problems Measures, Pittsburgh Sleep Diary, Pittsburgh Sleep Quality Index, Self-Rated Sleep Questionnaire and the Sleep Dissatisfaction Questionnaire; Functional Outcomes of Sleep Questionnaire, Quality of Life in Insomniacs and the Sleep-Wake Activity Inventory, Medical Outcomes Study—Sleep Problems Measures and Pittsburgh Sleep Quality Index. Administration can be oral, inhalation, nasal, mucosal, injection, infusion, or any combination thereof. Parameters of sleep such as REM and various stages of sleep can be measured using polysomnography, electroencephalography, sleep latancy, Bispectral Index (see, e.g., Lucey et al (2016) J. Sleep. Res. 25:625-635; Vacas et al (2016) Anesth. Analg. 123:206-212).

(2) Neuroprotection. Neuroprotection encompasses one or more of protection against seizures, epilepsy, neurotoxicity, mechanical trauma, and neuronal injury. Methods for assessing neuroprotection are available. See, e.g., Maas et al (2006) Lancet Neurol. 5:38-45; Hukkelboven et al (2005) 22:1025-1039; Dijkers (1997) J. Head Trauma Rehab. 12:74-91; Rogawski (1993) Trends Pharmacol. Sci. 14:325-331; McIntosh (1993) J. Neurotrauma. 10:215-243). These methods include Barthel index and Glasgow outcome scale.

(3) Anorexia (anorectant). The effects of anorectic agents administered to human subjects can be assessed, for example, by Three-Factor Eating Questionnaire (TFEQ-R18, TFEQ-R21), Dutch Eating Behavior Questionnaire, and Eating Disorders Inventory (see, Cappelleri et al (2009) Int. J. Obesity. 33:611-620; Kim et al (2014) Eat. Behavior. 15:87-90; Makris et al (2004) Appetite. 42:185-195. Administration can be oral, inhalation, nasal, mucosal, injection, infusion, or any combination thereof.

Questionnaires and Patient-Reported Outcomes

Questionnaires for assessing the psychological influences or medical influences of cannabinoids, are available. See, for example, Porter et al (2013) Report of a parent survey of cannabidiol-enriched cannabis use in pediatric treatment-resistant epilepsy. Epilepsy Behav. 29:574-577; Gorelick et al (2013) Around-the-clock oral THC effects on sleep in male chronic daily cannabis smokers. Am. J. Addict. 22:510-514; Trigo et al (2016) Effects of fixed or self-titrated dosages of Sativex on cannabis withdrawal and cravings. Drug Alcohol Depend. 161:298-306; and Ramesh et al (2013). Marijuana's dose-dependent effects in daily marijuana smokers. Experimental and Clinical Psychopharmacology. 21:287-293.

NMDA Receptors Assays and Other Assays

The present disclosure encompasses the lab techniques for measuring neuroprotection, (1) NMDA receptor binding assays; (2) Block NMDA toxicity in tissue culture; (3) Protect against hypobaric anemia in mice; (4) Improve “closed head injury” in rats; (5) Middle cerebral artery occlusion; (6) Four vessel occlusion in rats, 7) Cell culture tests to assess influence of cannabinoids on NMDA-induced cell toxicity using neuronal cells and glial cells and head trauma tests, as disclosed by Mechoulan U.S. Pat. No. 6,096,740, which is incorporated herein by reference in its entirety. Filbert et al provides methods of assessing neuroprotection, using hemotoxylin and eosin histology, which can reveal, for example, reduction in piriform cortical neuronal damage (Filbert et al (1999) Ann. NY Acad. Sci. 890:505-514). Radwan et al provide “mouse tetrad assay” to measure “locomotor activity, catalepsy, body temperature, and nociception” (Radwan, ElSohly, El-Alfy, Ahmed (2015) Isolation and pharmacological evaluation of minor cannabinoids from high-potency Cannabis sativa. J. Natural Products. 78:1271-1276). Mouse tetrad assay is described by Pertwee R G (2005) Pharmacological Actions of Cannabinoids. 168:1-51. Radwan et al, supra, uses binding assays to CB-1 receptor and to CB-2 receptor, according to Husni, McCurdy, Radwan et al (2014) J. Med. Chem. Res. 23:4295-4300. Seizure latancy assays and convulsion duration assays are detailed by Feigenbaum et al (1989) Proc. Nat. Acad. Sci. 86:9584-9587).

The present disclosure provides an ingredient with activity at cannabinoid receptors that allows therapeutic properties similar to those of delta-9-THC without the associated psychoactivity. These ingredients encompass an anorectant, an anti-epileptic agent, a modulator of inflammation, a neuroprotectant ingredient (dexanabinol [HU-211] same expected NMDA antagonist activity), an anti-encephalopathy agent in combination with CBD, an anti-glaucoma agent (reduced psychoactivity due to delta-8 THC content vs. delta-9). Also provided is a delta-9-THC modulator that increases the duration of delta-9-THC effects, or that modifies the characteristics of delta-9-THC activity. What can be modified is increased duration of delta-9-THC activity, such as increase in duration of at least 20%, at least 50%, at least 100% (twice as long), at least 150%, at least 200%, and so on. The present disclosure provides compositions for use as an ingredient in non-intoxicating cannabis products, such as non-alcoholic beer.

The present invention is not to be limited by compositions, reagents, methods, diagnostics, laboratory data, and the like, of the present disclosure. Also, the present invention is not be limited by any preferred embodiments that are disclosed herein.

What is claimed is:

1. A composition comprising the combination of delta-8-THC and a non-cannabinoid natural product:
(i) Wherein the non-cannabinoid natural product is capable of increasing the duration of the psychoactive or the non-psychoactive medicinal effects of delta-8-THC, as determinable by co-administering the delta-8-THC with or without the non-cannabinoid natural product, or
(ii) Wherein the non-cannabinoid natural product is capable of increasing the duration of the psychoactive or the non-psychoactive medicinal effects of delta-9-THC, as determinable by co-administering the delta-9-THC with or without the non-cannabinoid natural product, or
(iii) Wherein the non-cannabinoid natural product is capable of increasing the concentration of 11-hydroxy-delta-8-THC in the bloodstream of a human subject, as determinable by co-administering delta-8-THC with or without the non-cannabinoid natural product, or
(iv) Wherein the non-cannabinoid natural product is capable of increasing the concentration of 11-hydroxy-delta-9-THC in the bloodstream as determinable by co-administering delta-9-THC with or without the non-cannabinoid natural product to the human subject.

2. The composition of claim 1 that further comprises delta-9-THC.

3. The composition of claim 1 that does not comprise delta-9-THC.

4. The composition claim 1, wherein the cannabinoid and non-cannabinoid natural product are mixed together as a pharmaceutically acceptable composition for oral administration,
where optionally the pharmaceutically acceptable composition for oral administration is a powder, tablet, pill, capsule, slurry, suspension, or liquid composition.

5. The composition of claim 1, wherein the delta-8-THC and non-cannabinoid natural product that are not mixed together,
wherein the delta-8-THC is a component of a first pharmaceutically acceptable composition for oral administration, and
wherein the non-cannabinoid is a component of a second pharmaceutically acceptable composition for oral administration.

6. The composition of claim 1, that further comprises an inhibitor of at least one UDP-glucuronosyl transferase (UGT), wherein the UGT in absence of inhibitor is capable of catalyzing glucuronidation of one or both 11-hydroxy-delta-8-THC and 11-hydroxy-delta-9-THC,
where optionally the inhibitor is a substrate of UGT that is capable of acting as a competitive inhibitor of the at least one UGT.

7. The composition of claim 1, that further comprises an inhibitor of a cytochrome P450 enzyme (CYP enzyme),
wherein the CYP enzyme catalyzes the metabolism of a psychoactive cannabinoid to a non-psychoactive metabolite, or
wherein the CYP enzyme catalyzes the metabolism of a non-psychoactive medically active cannabinoid to a non-psychoactive non-medically active metabolite.

8. A method for administering the composition of claim 1 to a human subject, comprising the steps of:
(i) Providing said composition to the human subject,
(ii) Administering said composition to the human subject, or self-administering said composition by the human subject,
(iii) Allowing a cannabinoid of the composition to increase in concentration in the bloodstream of said human subject, and
(iv) Wherein said administering results in a psychological or medical influence on said human subject, assessing the influence by one or both of a questionnaire or a biochemical test.

9. A pharmaceutically acceptable composition capable of oral administration to a human subject, the composition comprising delta-8-THC and delta-9-THC, wherein
(i) The administered composition results in stimulation of CB1, or
(ii) The administered composition results in stimulation of CB2, or
(iii) The administered composition results in stimulation of CB1 to a greater extent than administration of delta-8-THC alone, or
(iv) The administered composition results in stimulation of CB1 to a greater extent than administration of delta-9-THC alone, or
(v) The administered composition results in stimulation of CB2 to a greater extent than administration of delta-8-THC alone, or
(vi) The administered composition results in stimulation of CB2 to a greater extent than administration of delta-9-alone,
(vii) The delta-8-THC in the administered composition enhances the pharmacological activity of the delta-9-THC in the administered composition, or
(viii) The delta-9-THC in the administered composition enhances the pharmacological activity of the delta-8-THC in the administered composition.

10. The pharmacologically acceptable composition of claim 9, that comprises a tablet containing delta-8-THC and delta-9-THC in the exact amounts of and, optionally, in about amounts, of:
(i) 10 mg of delta-8-THC and 10 mg of delta-9-THC, or
(ii) 5 mg delta-8-THC and 5 mg delta-9-THC, or
(iii) 2 mg delta-8-THC and 2 mg delta-9-THC, or
(iv) 1 mg delta-8-THC and 1 mg delta-9-THC, or
(v) 5 mg delta-8-THC and 2 mg delta-9-THC, or
(vi) 5 mg delta-8-THC and 1 mg delta-9-THC, or
(vii) 5 mg delta-8-THC and 0.5 mg delta-9-THC, or
(viii) 2 mg delta-8-THC and 1 mg delta-9-THC, or
(ix) 2 mg delta-8-THC and 0.5 mg delta-9-THC, or
(x) 2 mg delta-8-THC and 0.25 mg delta-9-THC, or
(xi) 1 mg delta-8-THC and 1 mg delta-9-THC, or
(xii) 1 mg delta-8-THC and 0.5 mg delta-9-THC, or
(xiii) 1 mg delta-8-THC and 0.25 mg delta-9-THC, or
(xiv) 10-30 mg of delta-8-THC and 10 mg of delta-9-THC, or
(xv) 10-30 mg of delta-8-THC and 5 mg of delta-9-THC, or
(xvi) 10-30 mg of delta-8-THC and 2 mg of delta-9-THC, or
(xvii) 10-30 mg of delta-8-THC and 0.5 mg of delta-9-THC, or
(xviii) Over 30 mg of delta-8-THC and 10 mg of delta-9-THC, or
(xix) Over 30 mg of delta-8-THC and 5 mg of delta-9-THC, or
(xx) Over 30 mg of delta-8-THC and 2 mg of delta-9-THC, or
(xxi) Over 30 mg of delta-8-THC and 0.5 mg of delta-9-THC.

11. The pharmaceutically acceptable composition of claim 9, that is capable of one or more of oral administration, intranasal administration, mucosal administration, topical administration, transdermal patch administration, or administration by inhaling, to a human subject.

12. The pharmaceutically acceptable composition of claim 9, wherein the stimulation of CB1 and the stimulation of CB2 in human subjects is determinable by administering to an animal subject a composition comprising delta-8-THC and delta-9-THC, by administering delta-8-alone, and by administering delta-9-alone, and by extrapolating the stimulation results to humans.

13. A method for administering the composition of claim 9 to a human subject, comprising the steps of:
(i) Providing said composition to the human subject,
(ii) Administering said composition to the human subject, or self-administering said composition by the human subject,
(iii) Allowing a cannabinoid of the composition to increase in concentration in the bloodstream of said human subject, and
(iv) Wherein said administering results in a psychological or medical influence on said human subject, assessing the influence by one or both of a questionnaire or a biochemical test.

14. A method for administering a non-cannabinoid natural product to a mammal, wherein the non-cannabinoid natural product is capable of increasing the concentration of a biologically active cannabinoid in a biological fluid of a test mammal,
or wherein the non-cannabinoid natural product is capable of reducing the conversion of a biologically active cannabinoid to a biologically inactive cannabinoid in the mammal,
the method comprising:
(i) The step of administering delta-8-THC to the mammal,
(ii) The step of administering the non-cannabinoid natural product to the mammal, where a first period of time is required to initiate and complete administering of the delta-8-THC, and where a second period of time is required to initiate and complete administering the non-cannabinoid natural product,
(iii) Wherein the first period of time is identical to the second period of time, or wherein the first period of time overlaps but is not identical to the second period of time, or wherein the first period of time does not overlap the second period of time,
(iv) The step where after the completion of both the first period of time and the second period of time, and within five days of completion of both the first period of time and the second period of time, taking at least one sample of the biological fluid from the test mammal and transferring the sample to a container,
(v) The step of subjecting the sample to a detection method that is capable of detecting one or more of the biologically active compounds delta-8-THC, 11-hydroxy-delta-8-THC, delta-9-THC, 11-hydroxy-delta-9-THC, 7-hydroxy-delta-8-THC, 7-hydroxy-delta-9-THC, or that is capable of detecting one or more biologically inactive compounds, 11-nor-9-carboxy-delta-8-THC, 11-nor-9-carboxy-delta-9-THC, 7-hydroxy-delta-8-THC, or 7-hydroxy-delta-9-THC,
(vi) The step of detecting said one or more biologically active compounds and biologically inactive compounds and calculating the concentration of said one or more compounds in the biological fluid.

15. The method of claim 14, wherein the non-cannabinoid natural product is:
(i) Capable of increasing the concentration of a biologically active cannabinoid in a biological fluid of a test mammal, as determinable by in vitro assays of cytochrome P450 enzymes, by in vitro assays UDP-glucuronosyl transferase (UGT) assays, or by in vivo tests with a mammalian subject,
(ii) Capable of reducing the conversion of a biologically active cannabinoid to a biologically inactive cannabinoid in the mammal, as determinable by in vitro assays of cytochrome P450 enzymes, by in vitro assays UDP-glucuronosyl transferase (UGT) assays, or by in vivo tests with a mammalian subject.

16. A composition comprising one or more of delta-8-THC, cannabidiol (CBD), delta-7-THC, delta-10-THC, or a cannabinoid where a double bond is present at a ring carbon other than at the 8-position or 9-position, wherein the composition provides an amount of delta-9-THC that is equal or less than a defined maximal amount of delta-9-THC, and wherein:
(i) The composition comprises delta-9-THC; or
(ii) The composition comprises a non-cannabinoid natural product that is capable of modulating the activity of a cytochrome P450 (CYP) enzyme in a human subject resulting in a CYP enzyme with modulated activity, and wherein the modulated activity results in increased in vivo concentrations in the human subject of an active metabolite of the administered delta-8-THC, cannabidiol (CBD), delta-7-THC, or delta-10-THC, or other similar THC isomer; or
(iii) The composition comprises a non-cannabinoid natural product that is capable of inhibiting the activity of UDP-glucuronosyl transferase (UGT), and wherein the inhibited UGT results in increased in vivo concentrations in the human subject of an active metabolite of the administered delta-8-THC, cannabidiol (CBD), delta-7-THC, or delta-10-THC, or other similar THC isomer; or
(iv) The cannabinoid where a double bond is present at a ring carbon other than at the 8-position or 9-position is not delta-7-THC or delta-10-THC, but still yields an active metabolite, and where the double bond at the ring carbon other than at the 8-position or 9-position is between carbons 9 and 11 (double bond on 11-methyl), carbons 7 and 6a, carbons 10 and 10a, and carbons 6a and 10a.

17. The composition of claim 16, wherein said active metabolite is one or more of psychoactive, medically active, and pharmacologically active.

18. The composition of claim 16, wherein the defined maximal concentration of delta-9-THC is defined by one or both of: (i) law by the State of Washington, the State of Oregon, the State of California, or the State of Colorado, or any other states or jurisdictions with similarly defined laws, or (ii) Drug testing policy by the National Football League or other professional or non-professional sport governing bodies.

19. The composition of claim 16, wherein the defined maximal concentration of delta-9-THC, or its signaling metabolites, is an amount detectable in whole blood, in blood plasma, in urine, or in other bodily fluid, of the human subject.

20. The composition of claim 16, comprising one or more of delta-8-THC, cannabidiol (CBD), delta-7-THC, or delta-10-THC,
wherein the delta-7-THC possesses psychoactive or medicinal activity and wherein said activity is exerted by 11-hydroxy-delta-7-THC, or
where in the delta-10-THC possesses psychoactive or medicinal activity, and wherein said activity is exerted by 11-hydroxy-delta-10-THC, or
wherein other similar isomers possess psychoactive or medicinal activity, and wherein said activity is exerted by the mono-hydroxy metabolites of such isomers.
